(19) United States
(12) Patent Application Publication
(10) Pub. No.: US 2022/0214252 A1
(43) Pub. Date: Jul. 7, 2022

(54) METHODS, DEVICES, AND APPARATUS FOR DISPENSING AND ASPIRATING LIQUIDS ON ARRAY PLATES

(71) Applicant: CURIOX BIOSYSTEMS PTE LTD., Singapore (SG)

(72) Inventors: Namyong KIM, Boston, MA (US); Kong Leong CHEN, Singapore (SG)

(21) Appl. No.: 17/612,174

(22) PCT Filed: May 15, 2020
(86) PCT No.: PCT/US2020/033251

§ 371 Date: Nov. 17, 2021

Related U.S. Application Data
(60) Provisional application No. 62/849,694, filed on May 17, 2019.

(51) Int. Cl. G01N 1/34; B01L 3/02

(52) U.S. Cl. CPC G01N 1/34; G01N 35/1016; B01L 3/0203

(57) ABSTRACT

A method includes dispensing a liquid from a dispenser having a first piston channel and a first nozzle channel that is non-parallel to the first piston channel and is connected to the first piston channel at a first intersection. The dispenser includes a first piston configured to slide within the first piston channel; a first nozzle coupled with the first nozzle channel; a first valve located between a dispensing-liquid reservoir and the first intersection; and a second valve located between the first intersection and the first nozzle. Dispensing the liquid from the first dispenser includes pulling the first piston to initiate flow of a liquid in the dispensing-liquid reservoir to the first intersection; and subsequent to pulling the first piston, pushing the first piston to initiate flow of the liquid in the first intersection to the first nozzle so that the liquid is dispensed from the first nozzle.

### TECHNICAL FIELD

The disclosed embodiments relate generally to methods, devices, and apparatus for washing samples (e.g., cells, particles, etc.). More particularly, the disclosed embodiments relate to methods, devices, and apparatus for washing samples on array plates and slides.

### BACKGROUND

An array plate is also called a microtiter plate, microplate, or microwell plate. Array plates are typically used to hold respective liquid droplets separately for biological and/or chemical reaction. For example, a well-type array plate includes a plurality of wells so that each liquid droplet or each sample may be dispensed into a separate well for further processing. Typically, the number of wells is selected from 6, 24, 96, 384, 1536, 3456, and 9600.

Samples (e.g., cells) are frequently washed in biological and/or chemical assays or operations. Washing typically involves adding a wash solution to a sample solution, including samples (e.g., cells), on the slide and removing the mixture of the wash solution and the sample solution. By repeating the dilution and partial removal of the sample solution, the concentration of chemicals and/or biological reagents other than the samples are reduced. However, variations in the sample washing increase measurement errors, which are not desirable for accurate assays.

In addition, certain cells (e.g., suspension cells, non-adherent cells, and weakly adherent cells) do not strongly adhere to the slide. Thus, during removal of the mixture, cells may be removed along with the mixture, thereby reducing the number of cells that remain on the hydrophilic area of the slide after the washing. Because a reliability of cell-based reactions typically requires a sufficient number of cells, the loss of cells during washing negatively affects cell-based reactions.

### SUMMARY

Accordingly, there is need for methods, devices, and apparatus that provide improved accuracy and reduced time in washing cells. Such methods, devices, and apparatus plates may replace the conventional methods, devices, and apparatus for washing cells. In addition, such methods, devices, and apparatus may better retain cells during washing, and reduce or eliminate the loss of cells during washing, thereby improving the reliability of cell-based reactions. Similarly, such methods, devices, and apparatus may be used in washing other types of samples, such as beads or particles conjugated with target molecules.

A number of embodiments that overcome the limitations and disadvantages of existing methods, devices, and apparatus are presented in more detail below. These embodiments provide methods, devices, and apparatus for washing a sample in a solution.

As described in more detail below, in accordance with some embodiments, a method includes dispensing a liquid from a first dispenser. The first dispenser has a first piston channel and a first nozzle channel that is non-parallel to the first piston channel and is connected to the first piston channel at a first intersection. The first dispenser includes a first piston configured to slide at least partially within the first piston channel; a first nozzle coupled with the first nozzle channel; a first valve located between a dispensing-liquid reservoir and the first intersection, the first valve configured to allow a liquid in the dispensing-liquid reservoir to flow to the first intersection through the first valve and prevent a liquid in the first intersection from flowing to the dispensing-liquid reservoir through the first valve; and a second valve located between the first intersection and the first nozzle, the second valve configured to allow a liquid in the first intersection to flow to the first nozzle through the second valve and prevent a liquid in the first nozzle to flow to the first intersection through the second valve. Dispensing the liquid from the first dispenser includes pulling the first piston to initiate flow of a liquid in the dispensing-liquid reservoir to the first intersection; and, subsequent to pulling the first piston, pushing the first piston to initiate flow of the liquid in the first intersection to the first nozzle so that the liquid is dispensed from the first nozzle.

In some embodiments, the method also includes dispensing a liquid from a second dispenser, the second dispenser having a second piston channel and a second nozzle channel that is non-parallel to the second piston channel and is connected to the second piston channel at a second intersection. The second dispenser includes a second piston configured to slide at least partially within the second piston channel; a second nozzle coupled with the second nozzle channel; a third valve located between the dispensing-liquid reservoir and the second intersection, the third valve configured to allow the liquid in the dispensing-liquid reservoir to flow to the second intersection through the third valve and prevent a liquid in the second intersection from flowing to the dispensing-liquid reservoir through the third valve; and a fourth valve located between the second intersection and the second nozzle, the fourth valve configured to allow a liquid in the second intersection to flow to the second nozzle through the fourth valve and prevent a liquid in the second nozzle to flow to the second intersection through the fourth valve. Dispensing the liquid from the second dispenser includes pulling the second piston to initiate flow of the liquid in the dispensing-liquid reservoir to the second intersection; and, subsequent to pulling the second piston, pushing the second piston to initiate flow of the liquid in the second intersection to the second nozzle so that the liquid is dispensed from the second nozzle.

In some embodiments, the method includes concurrently pulling the first piston and the second piston at a same first speed to concurrently initiate the flow of the liquid in the dispensing-liquid reservoir to the first intersection and the second intersection; and, concurrently pushing the first piston and the second piston at a same second speed to concurrently initiate the flow of the liquid in the first intersection to the first nozzle and the flow of the liquid in the second intersection to the second nozzle.

In some embodiments, the method further includes aspirating a liquid with a first aspirator having a third piston channel and a third nozzle channel that is non-parallel to the third piston channel and is connected to the third piston channel at a third intersection. The first aspirator includes a third piston configured to slide at least partially within the third piston channel; a third nozzle coupled with the third nozzle channel; a fifth valve located between an aspirated-liquid reservoir and the third intersection, the fifth valve configured to allow a liquid in the third intersection to flow to the aspirated-liquid reservoir through the fifth valve and prevent a liquid in the aspirated-liquid reservoir from flowing to the third intersection through the fifth valve; and a sixth valve located between the third intersection and the third nozzle, the sixth valve configured to allow a liquid in the third nozzle to flow to the third intersection through the sixth valve and prevent a liquid in the third intersection from flowing to the third nozzle through the sixth valve. Aspirating the liquid with the first aspirator includes pulling the third piston to initiate flow of the liquid from the third nozzle to the third intersection; and, subsequent to pulling the third piston, pushing the third piston to initiate flow of the liquid in the third intersection to the aspirated-liquid reservoir.

In some embodiments, the method further includes aspirating a liquid with a second aspirator having a fourth piston channel and a fourth nozzle channel that is non-parallel to the fourth piston channel and is connected to the fourth piston channel at a fourth intersection. The second aspirator includes a fourth piston configured to slide at least partially within the fourth piston channel; a fourth nozzle coupled with the fourth nozzle channel; a seventh valve located between the aspirated-liquid reservoir and the fourth intersection, the seventh valve configured to allow a liquid in the aspirated-liquid reservoir to flow to the fourth intersection through the seventh valve and prevent a liquid in the fourth intersection from flowing to the aspirated-liquid reservoir through the seventh valve; and an eighth valve located between the fourth intersection and the fourth nozzle, the eighth valve configured to allow a liquid in the fourth intersection to flow to the fourth nozzle through the eighth valve. Aspirating the liquid with the second aspirator includes pulling the fourth piston to initiate flow of the liquid from the fourth nozzle to the fourth intersection; and, subsequent to pulling the fourth piston, pushing the fourth piston to initiate flow of the liquid in the fourth intersection to the aspirated-liquid reservoir.

In some embodiments, the method also includes concurrently pulling the third piston and the fourth piston at a same first speed to concurrently initiate the flow of the liquid in the dispensing-liquid reservoir to the first intersection and the second intersection; and, concurrently pushing the third piston and the fourth piston at a same second speed to concurrently initiate the flow of the liquid in the first intersection to the first nozzle and the flow of the liquid in the second intersection to the second nozzle.

In some embodiments, the first piston channel is substantially perpendicular to the first nozzle channel.

In accordance with some embodiments, a method includes aspirating a liquid with a first aspirator having a first piston channel and a first nozzle channel that is non-parallel to the first piston channel and is connected to the first piston channel at a first intersection. The first aspirator includes a first piston configured to slide at least partially within the first piston channel; a first nozzle coupled with the first nozzle channel; a first valve located between an aspirated-liquid reservoir and the first intersection, the first valve configured to allow a liquid in the first intersection to flow to the aspirated-liquid reservoir through the first valve and prevent a liquid in the aspirated-liquid reservoir from flowing to the first intersection through the first valve; and a second valve located between the first intersection and the first nozzle, the second valve configured to allow a liquid in the first nozzle to flow to the first intersection through the second valve and prevent a liquid in the first intersection from flowing to the first nozzle through the second valve. Aspirating the liquid with the first aspirator includes pulling the first piston to initiate flow of the liquid from the first nozzle to the first intersection; and, subsequent to pulling the first piston, pushing the first piston to initiate flow of the liquid in the first intersection to the aspirated-liquid reservoir.

In some embodiments, the method further includes aspirating a liquid with a second aspirator having a second piston channel and a second nozzle channel that is non-parallel to the second piston channel and is connected to the second piston channel at a second intersection. The second aspirator includes a second piston configured to slide at least partially within the second piston channel; a second nozzle coupled with the second nozzle channel; a third valve located between the aspirated-liquid reservoir and the second intersection, the third valve configured to allow a liquid in the aspirated-liquid reservoir to flow to the second intersection through the third valve and prevent a liquid in the second intersection from flowing to the aspirated-liquid reservoir through the third valve; and a fourth valve located between the second intersection and the second nozzle, the fourth valve configured to allow a liquid in the second intersection to flow to the second nozzle through the fourth valve. Aspirating the liquid with the second aspirator includes pulling the second piston to initiate flow of the liquid from the second nozzle to the second intersection; and, subsequent to pulling the second piston, pushing the second piston to initiate flow of the liquid in the second intersection to the aspirated-liquid reservoir.

In some embodiments, the method includes concurrently pulling the first piston and the second piston at a same first speed to concurrently initiate the flow of the liquid in the aspirated-liquid reservoir to the first intersection and the second intersection; and, concurrently pushing the first piston and the second piston at a same second speed to concurrently initiate the flow of the liquid in the first intersection to the first nozzle and the flow of the liquid in the second intersection to the second nozzle.

In accordance with some embodiments, a device includes a first dispenser defining a first piston channel and a first nozzle channel that is non-parallel to the first piston channel and is connected to the first piston channel at a first intersection. The first dispenser includes a first piston configured to slide at least partially within the first piston channel; a first nozzle coupled with the first nozzle channel; a first valve located between a dispensing-liquid reservoir and the first intersection, the first valve configured to allow a liquid in the dispensing-liquid reservoir to flow to the first intersection through the first valve and prevent a liquid in the first intersection from flowing to the dispensing-liquid reservoir through the first valve; and a second valve located between the first intersection and the first nozzle, the second valve configured to allow a liquid in the first intersection to flow to the first nozzle through the second valve and prevent a liquid in the first nozzle to flow to the first intersection through the second valve.

In some embodiments, the device also includes a second dispenser defining a second piston channel and a second nozzle channel that is non-parallel to the second piston channel and is connected to the second piston channel at a second intersection. The second dispenser includes a second piston configured to slide at least partially within the second piston channel; a second nozzle coupled with the second nozzle channel; a third valve located between the dispensing-liquid reservoir and the second intersection, the third valve configured to allow a liquid in the dispensing-liquid reservoir to flow to the second intersection through the third valve and prevent a liquid in the second intersection from flowing to the dispensing-liquid reservoir through the third valve; and a fourth valve located between the second intersection and the second nozzle, the fourth valve configured to allow a liquid in the second intersection to flow to the second nozzle through the fourth valve and prevent a liquid in the second nozzle to flow to the second intersection through the fourth valve.

In some embodiments, the first piston and the second piston are mechanically coupled to each other so that the first piston and the second piston are configured to move at a same speed in a same direction.

In some embodiments, the device further includes a first aspirator defining a third piston channel and a third nozzle channel that is non-parallel to the third piston channel and is connected to the third piston channel at a third intersection. The first aspirator includes a third piston configured to slide at least partially within the third piston channel; a third nozzle coupled with the third nozzle channel; a fifth valve located between an aspirated-liquid reservoir and the third intersection, the fifth valve configured to allow a liquid in the third intersection to flow to the aspirated-liquid reservoir through the fifth valve and prevent a liquid in the aspirated-liquid reservoir from flowing to the third intersection through the fifth valve; and a sixth valve located between the third intersection and the third nozzle, the sixth valve configured to allow a liquid in the third nozzle to flow to the third intersection through the sixth valve and prevent a liquid in the third intersection from flowing to the third nozzle through the sixth valve.

In some embodiments, the device further includes a second aspirator defining a fourth piston channel and a fourth nozzle channel that is non-parallel to the fourth piston channel and is connected to the fourth piston channel at a fourth intersection. The second aspirator includes a fourth piston configured to slide at least partially within the fourth piston channel; a fourth nozzle coupled with the fourth nozzle channel; a seventh valve located between the aspirated-liquid reservoir and the fourth intersection, the seventh valve configured to allow a liquid in the aspirated-liquid reservoir to flow to the fourth intersection through the seventh valve and prevent a liquid in the fourth intersection from flowing to the aspirated-liquid reservoir through the seventh valve; and an eighth valve located between the fourth intersection and the fourth nozzle, the eighth valve configured to allow a liquid in the fourth intersection to flow to the fourth nozzle through the eighth valve.

In some embodiments, the third piston and the fourth piston are mechanically coupled to each other so that the third piston and the fourth piston are configured to move at a same speed in a same direction.

In some embodiments, the first piston channel is substantially perpendicular to the first nozzle channel.

In accordance with some embodiments, a device includes a first aspirator defining a first piston channel and a first nozzle channel that is non-parallel to the first piston channel and is connected to the first piston channel at a first intersection. The first aspirator including a first piston configured to slide at least partially within the first piston channel; a first nozzle coupled with the first nozzle channel; a first valve located between an aspirated-liquid reservoir and the first intersection, the first valve configured to allow a liquid in the first intersection to flow to the aspirated-liquid reservoir through the first valve and prevent a liquid in the aspirated-liquid reservoir from flowing to the first intersection through the first valve; and a second valve located between the first intersection and the first nozzle, the second valve configured to allow a liquid in the first nozzle to flow to the first intersection through the second valve and prevent a liquid in the first intersection from flowing to the first nozzle through the second valve.

In some embodiments, the device further includes a second aspirator defining a second piston channel and a second nozzle channel that is non-parallel to the second piston channel and is connected to the second piston channel at a second intersection. The second aspirator includes a second piston configured to slide at least partially within the second piston channel; a second nozzle coupled with the second nozzle channel; a third valve located between the aspirated-liquid reservoir and the second intersection, the third valve configured to allow a liquid in the aspirated-liquid reservoir to flow to the second intersection through the third valve and prevent a liquid in the second intersection from flowing to the aspirated-liquid reservoir through the third valve; and a fourth valve located between the second intersection and the second nozzle, the fourth valve configured to allow a liquid in the second intersection to flow to the second nozzle through the fourth valve.

In some embodiments, the first piston and the second piston are mechanically coupled to each other so that the first piston and the second piston are configured to move at a same speed in a same direction.

### BRIEF DESCRIPTION OF THE DRAWINGS

For a better understanding of the aforementioned embodiments as well as additional embodiments, reference should be made to the Description of Embodiments below, in conjunction with the following drawings in which like reference numerals refer to corresponding parts throughout the figures.

FIGS. 1A-1F illustrate a washing operation with a conventional micro-titer plate.

FIGS. 2A-2E illustrate washing operations with an array plate having a hydrophilic region and a hydrophobic area in accordance with some embodiments.

FIGS. 3A-3G illustrate a washing operation in accordance with some embodiments.

FIGS. 4A-4C illustrate a dispenser and its components in accordance with some embodiments.

FIG. 4D illustrates a dispenser coupled with a reservoir in accordance with some embodiments.

FIG. 4E illustrates a set of multiple dispensers in accordance with some embodiments.

FIG. 4F illustrates coupled pistons in accordance with some embodiments.

FIGS. 4G and 4H illustrate a block with piston channels and nozzle channels in accordance with some embodiments.

FIGS. 5A-5D illustrate a dispensing operation in accordance with some embodiments.

FIGS. 6A-6E illustrate an aspiration operation in accordance with some embodiments.

FIG. 6F illustrates a set of multiple aspirators in accordance with some embodiments.

Like reference numerals refer to corresponding parts throughout the drawings.

### DESCRIPTION OF EMBODIMENTS

Methods, devices, and apparatus for washing samples are described. Reference will be made to certain embodiments, examples of which are illustrated in the accompanying drawings. While the claims will be described in conjunction with the embodiments, it will be understood that it is not intended to limit the claims to these particular embodiments alone. On the contrary, the embodiments are intended to cover alternatives, modifications and equivalents that are within the spirit and scope of the appended claims.

Moreover, in the following description, numerous specific details are set forth to provide a thorough understanding of the embodiments. However, it will be apparent to one of ordinary skill in the art that the embodiments may be practiced without these particular details. In other instances, methods, procedures, components, and networks that are well-known to those of ordinary skill in the art are not described in detail to avoid obscuring aspects of the embodiments.

It will also be understood that, although the terms first, second, etc. may be used herein to describe various elements, these elements should not be limited by these terms. These terms are only used to distinguish one element from another. For example, a first piston could be termed a second piston, and, similarly, a second piston could be termed a first piston, without departing from the scope of the embodiments. The first piston and the second piston are both pistons, but they are not the same piston.

The terminology used in the description of the embodiments herein is for the purpose of describing particular embodiments only and is not intended to be limiting of the invention. As used in the description of the embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

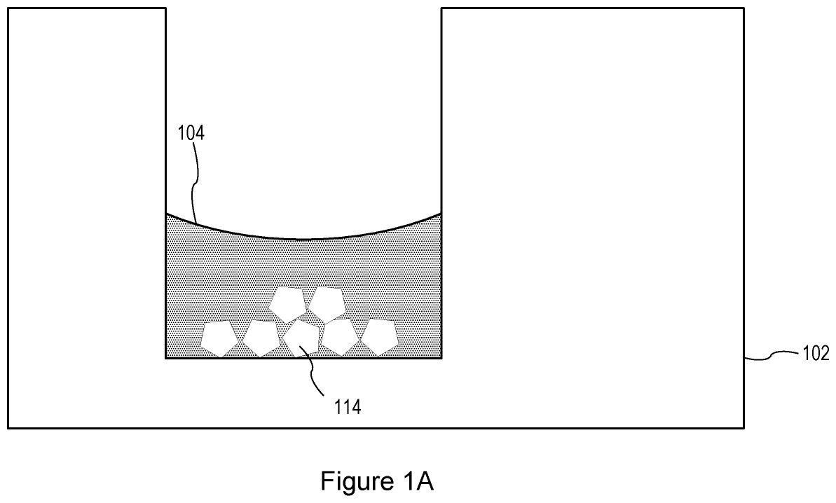

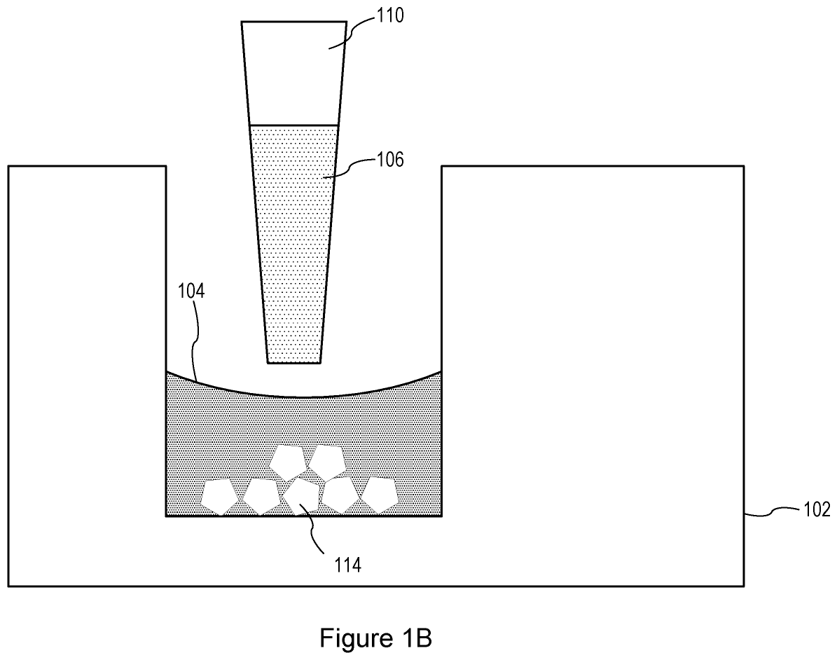

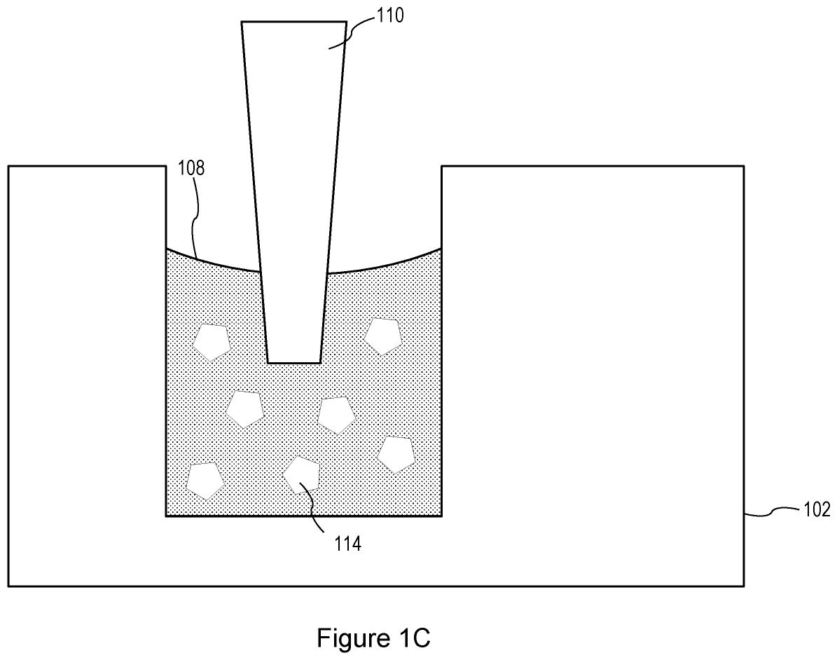

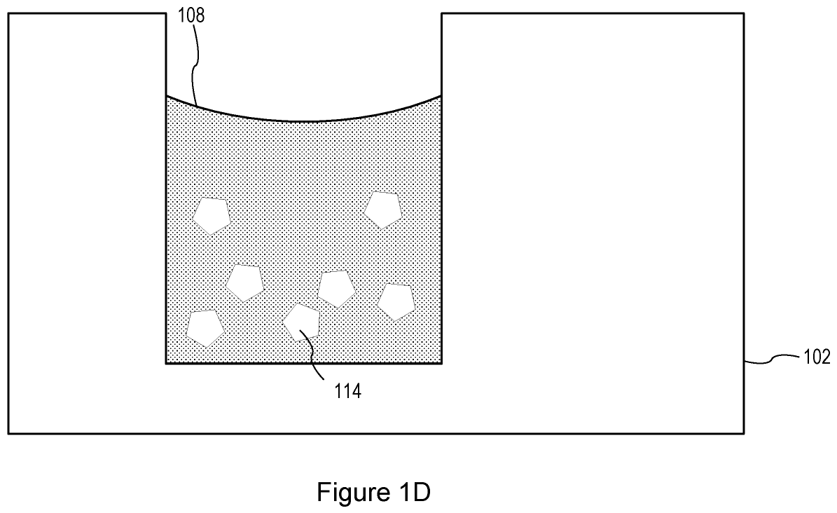

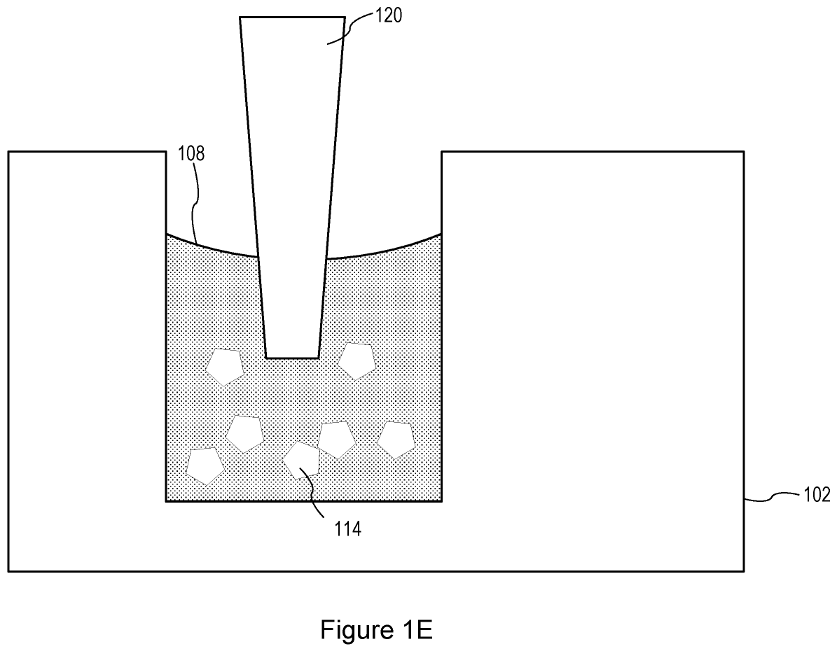

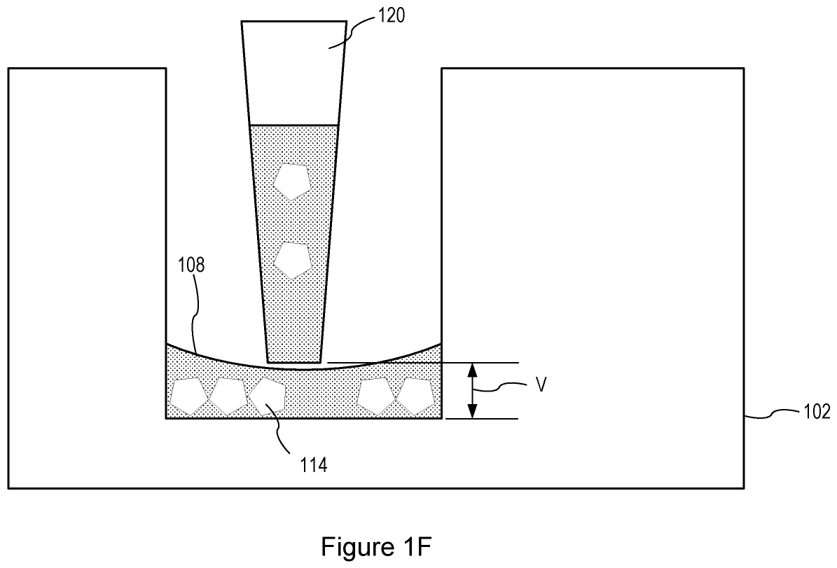

FIGS. 1A-1F illustrate a washing operation with a conventional micro-titer plate.

FIG. 1A illustrates solution 104 containing samples 114 (e.g., cells, particles, etc.) in a well that is defined in micro-titer plate 102.

FIG. 1B illustrates that dispenser 110 containing wash liquid 106 (e.g., a wash buffer, such as phosphate-buffered saline, Tris-buffered saline, borate-buffered saline, and TE buffer) is used for washing samples 114.

For example, as shown in FIG. 1C, wash liquid 106 in dispenser 110 is dispensed into solution 104, thereby forming mixture 108 (e.g., liquid) of solution 104 and wash liquid 106. As a result, chemical and biological reagents in solution 104 are diluted (e.g., concentrations of chemicals and biological reagents in solution 104 are reduced). FIG. 1C also illustrates that at least a portion of samples 114 is lift-off from the bottom of the well and suspended in mixture 108, due to the liquid flow caused by introduction of wash liquid 106 into solution 104.

FIG. 1D illustrates that samples 114 settle over time.

FIG. 1E illustrates that aspirator 120 is used to aspirate (e.g., remove) a portion of mixture 108.

FIG. 1F illustrates that aspirator 120 has aspirated a portion of mixture 108. The volume of mixture 108 remaining in the well defined in micro-titer plate 102, after the portion of mixture 108 is aspirated, is determined at least in part by height V of aspirator 120 (e.g., a distance between a nozzle tip of aspirator 120 and a bottom of the well defined in the micro-titer plate 102).

FIG. 1F also illustrates that a portion of samples 114 is also aspirated by aspirator 120. Wells of micro-titer plate 102 have a high aspect ratio (e.g., a ratio between the height of the well and the diameter of the well). Thus, once samples 114 are agitated, it takes a long time for samples 114 to settle down. If a portion of mixture 108 is aspirated before samples 114 have fully settled down, a portion of samples 114 that is aspirated is increased.

In addition, FIG. 1F illustrates that samples 114 cluster toward corners of the well when the volume of mixture 108 is reduced. In addition, mixture 108 clings toward corners of the well. Both of these can reduce the efficiency of washing.

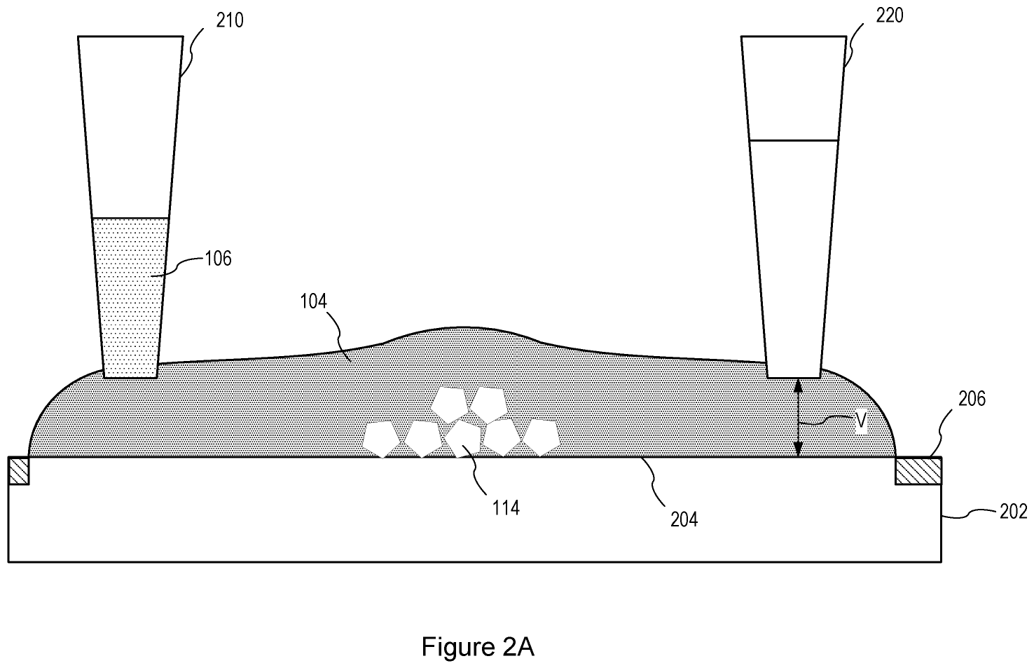

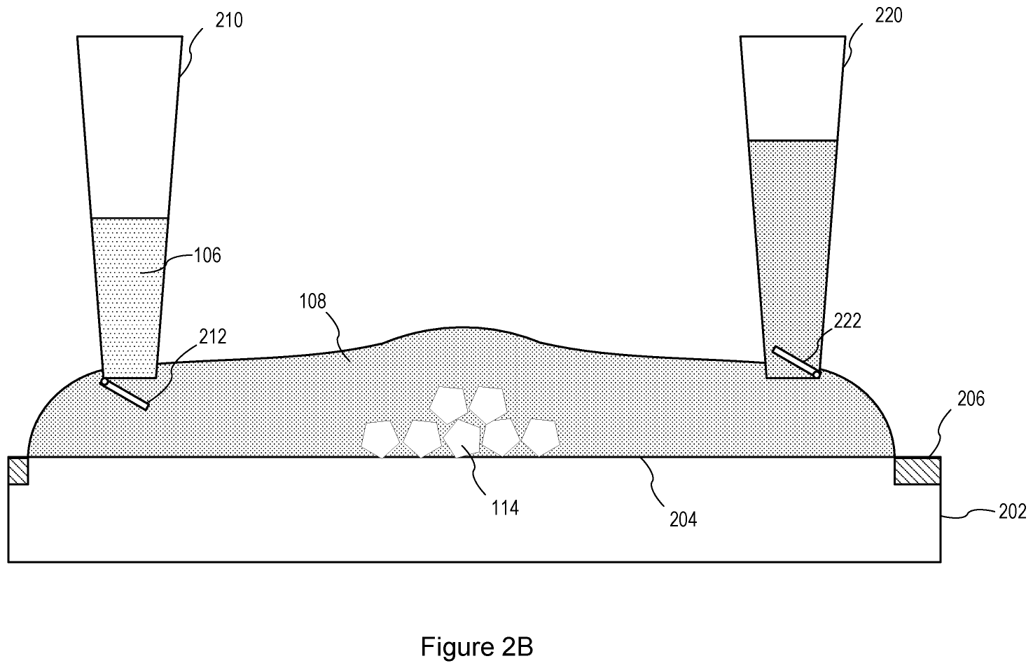

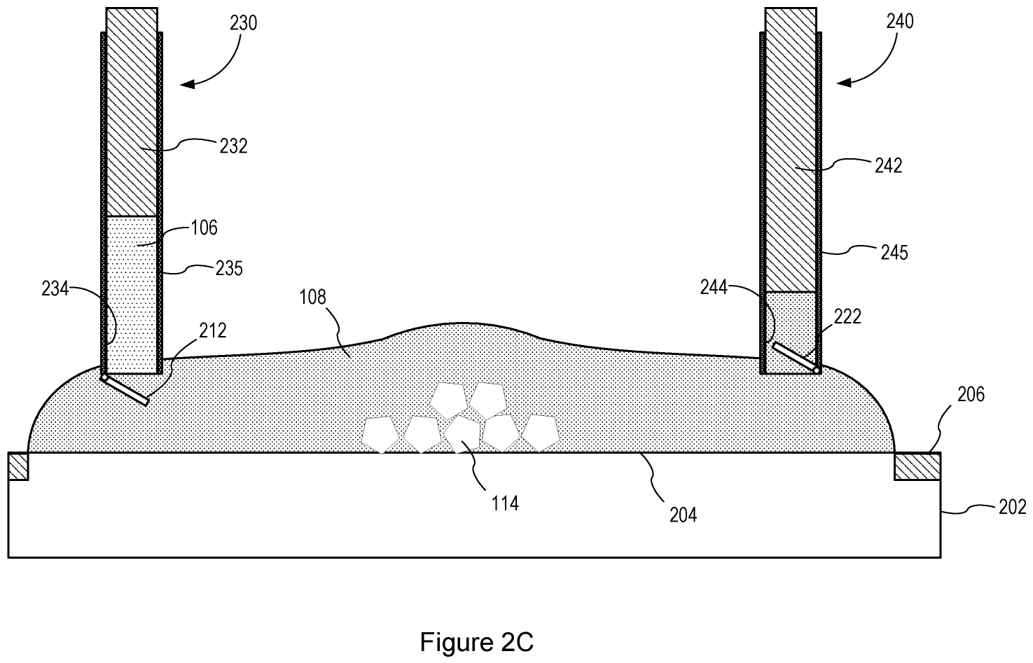

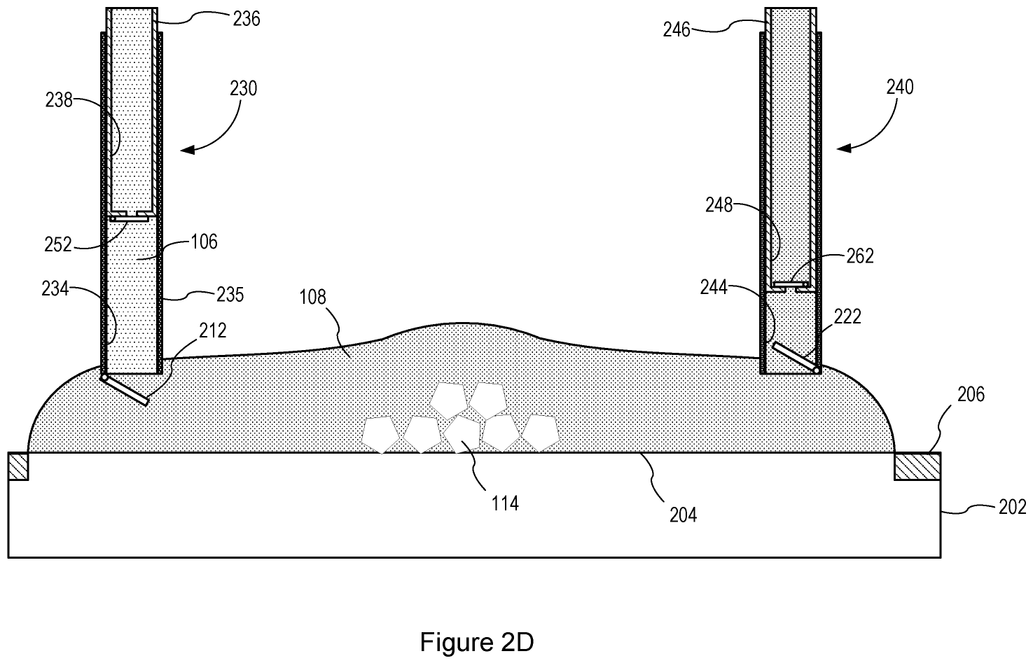

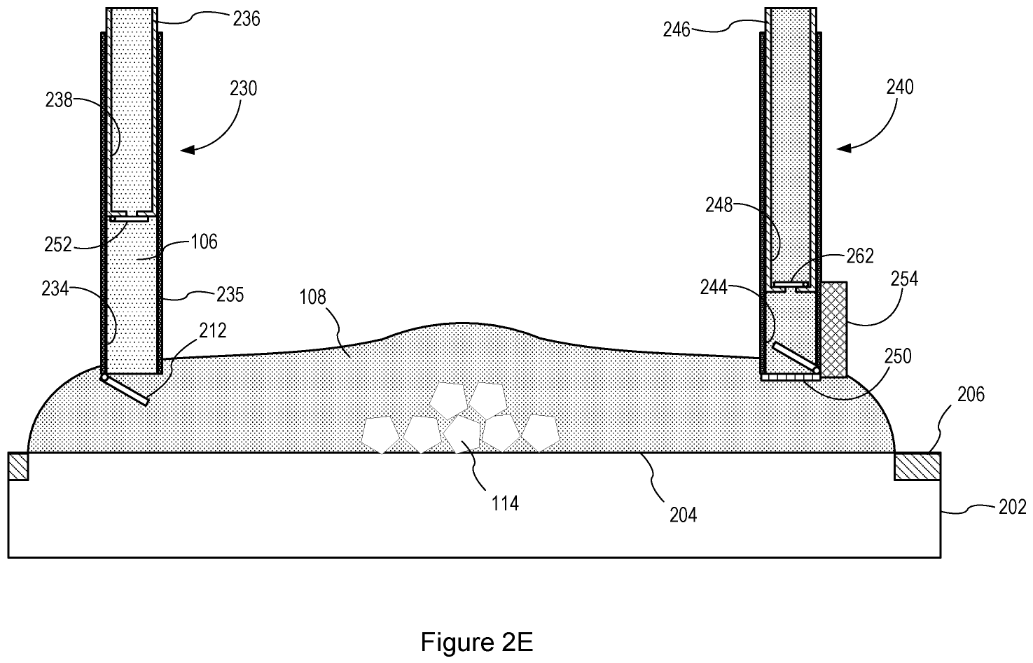

FIGS. 2A-2E illustrate washing operations with an array plate having a hydrophilic region and a hydrophobic area in accordance with some embodiments.

FIG. 2A is a partial cross-section of an array plate, where hydrophilic region 204 is surrounded by hydrophobic area 206. In FIG. 2A, solution 104 containing samples 114 is located over hydrophilic region 204. Solution 104 is retained over hydrophilic region 204, as surrounding hydrophobic area 206 prevents spreading of solution 104 beyond hydrophilic region 204.

FIG. 2A also illustrates dispenser 210 and aspirator 220. Dispenser 210 includes liquid 106 for washing samples 114 in solution 104 (by dilution of solution 104).

The array plate illustrated in FIG. 2A is configured to hold solution 104 without tall side walls, like conventional micro-titer plates. Thus, in the configuration shown in FIG. 2A, there are no corners toward which solution 104 and samples 114 cluster.

In addition, solution 104 in FIG. 2A has a low aspect ratio (e.g., a ratio between the height of solution 104 and the width or diameter of solution 104 on the array plate is less than the height of solution 104 and the diameter of solution 104 in a conventional micro-titer plate, sometimes by a factor of 2, 4, 6, 8, 10, or 20). Thus, when samples 114 are agitated, samples 114 in solution 104 on the array plate can settle faster than samples in solution 104 in a conventional micro-titer plate (shown in FIG. 1F).

In some embodiments, magnetic particles configured to couple with cells (e.g., coated with materials that can reversibly or irreversibly bind to the cells) are included in solution 104 (e.g., by introducing the magnetic particles into solution 104). Once the magnetic particles bind to the cells in solution 104, a magnetic field is applied to the magnetic particles in solution 104 to accelerate settling of the magnetic particles (and associated cells).

In some cases, the distance V between hydrophilic region 204 and aspirator 220 (e.g., a distance between hydrophilic surface 204 and a nozzle tip of aspirator 220) is important in improving retention of samples 114. In some embodiments, aspirator 220 needs to be positioned at least 100 μm from hydrophilic region 204. In some embodiments, aspirator 220 needs to be positioned at least 200 μm from hydrophilic region 204. In some embodiments, aspirator 220 needs to be positioned at least 300 μm from hydrophilic region 204.

FIG. 2B illustrate dispenser 210 and aspirator 220 with improved volume control. A variation in the dispensed volume and/or the aspirated volume contributes to a variation in the dilution factor, which leads to an increased error in assays. Thus, reducing the variation in the volume of the dispensed liquid and/or the volume of the aspirated liquid improves the assay accuracy (e.g., an accuracy of an assay performed using the washing operation).

In FIG. 2B, dispenser 210 includes valve 212 (e.g., a one-way valve, which is also called a check valve, or a check valve) to reduce the variation in the volume of the dispensed liquid, and aspirator 220 includes valve 222 (e.g., a one-way valve or a check valve) to reduce the variation in the volume of the aspirated liquid. For example, a respective valve allows a liquid to flow in one direction but prevents the liquid to flow in the opposite direction (e.g., valve 212 allows the liquid in dispenser 210 to exit from dispenser 210 through valve 212 but prevents a liquid to enter into dispenser 210 through valve 212, and valve 222 allows mixture 108 to enter into aspirator 220 through valve 222 but prevents mixture 108 in aspirator 220 from exiting from aspirator 220 through valve 222).

FIG. 2C is similar to FIG. 2B, except that dispenser 230 is used in place of dispenser 210 and aspirator 240 is used in place of aspirator 220. Dispenser 230 includes piston 232 (e.g., a plunger) configured to slide within channel 234 for dispensing wash liquid 106 in channel 234 through valve 212. Aspirator 240 includes piston 242 (e.g., a plunger) configured to slide within channel 244 for aspirating a liquid (mixture 108) into channel 244 through valve 222. In some embodiments, channel 234 is defined by tube 235. In some embodiments, channel 244 is defined by tube 245.

In some implementations, the volume of the aspirated liquid is controlled by a movement of piston 242 (e.g., a diameter of channel 244 and a travel distance of piston 242). In some embodiments, the diameter of piston 242 is less than the diameter of mixture 108, which facilitates an accurate control of the volume of the aspirated solution. Similarly, the volume of the aspirated liquid is accurately controlled by a movement of piston 232. In some implementations, the volume of the aspirated liquid (and/or the remaining liquid) is determined based on a height of an aspirator (e.g., a portion of the liquid located above the tip of aspirator 240 is aspirated and a portion of the liquid located below the tip of aspirator 240 remains, as shown in FIG. 1F).

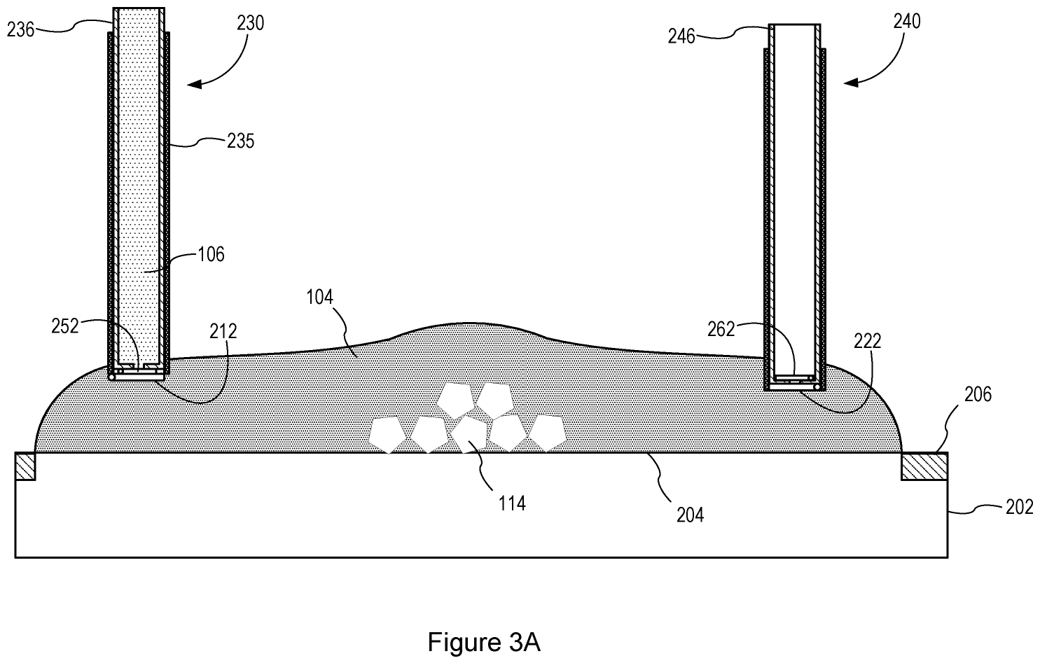

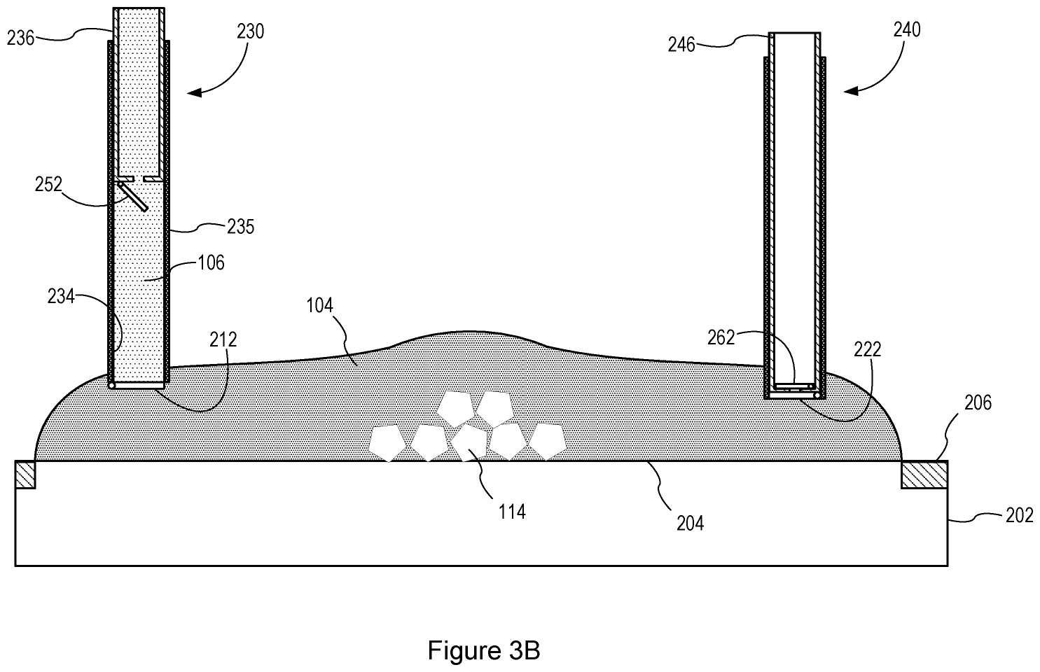

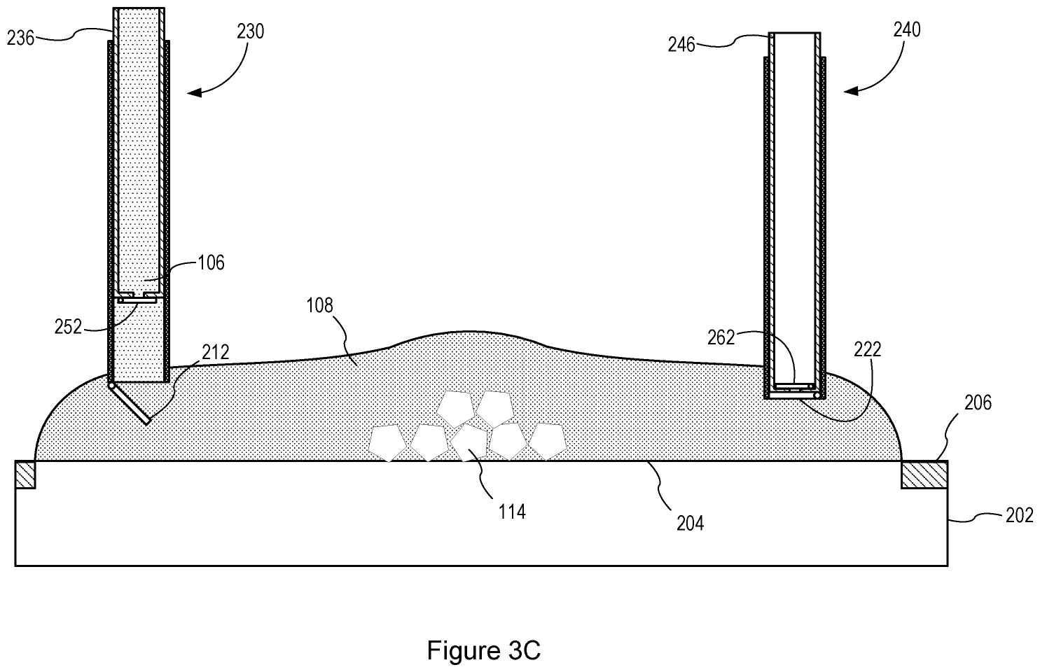

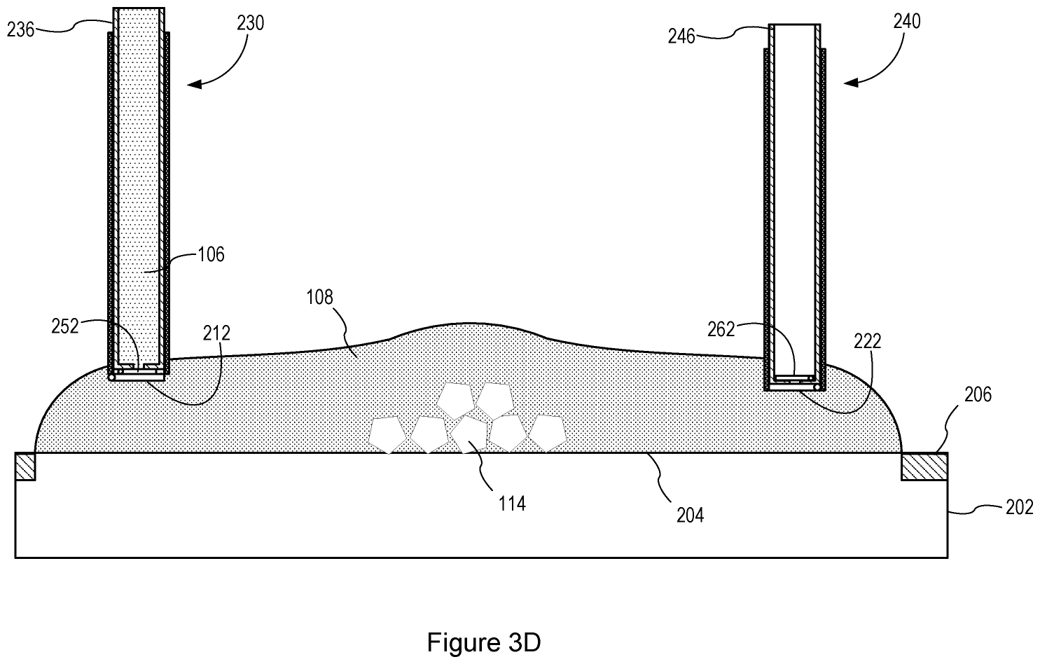

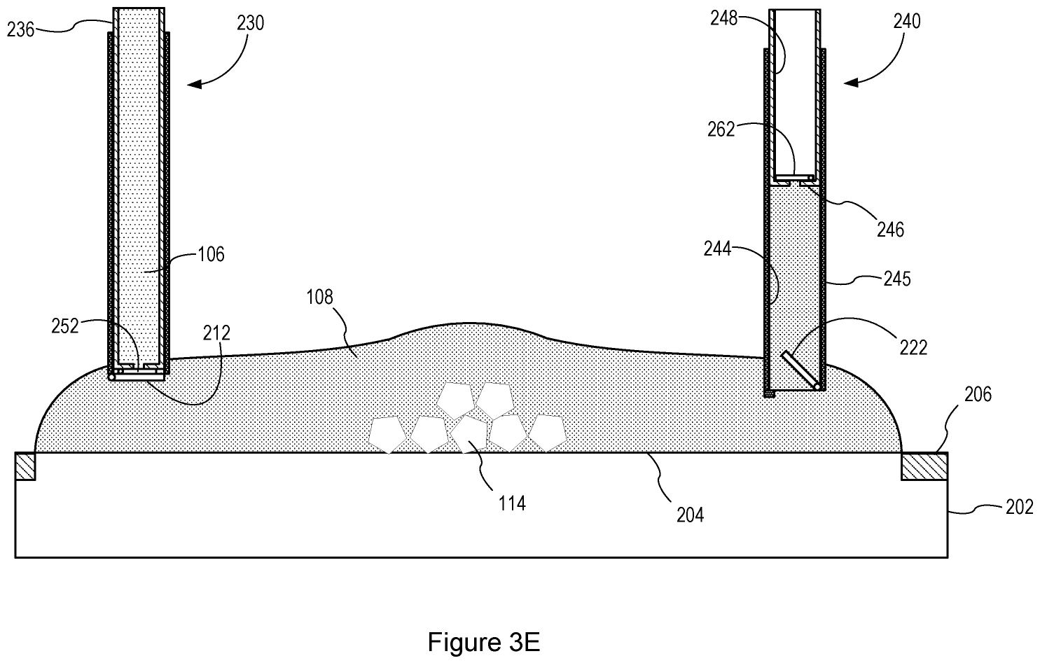

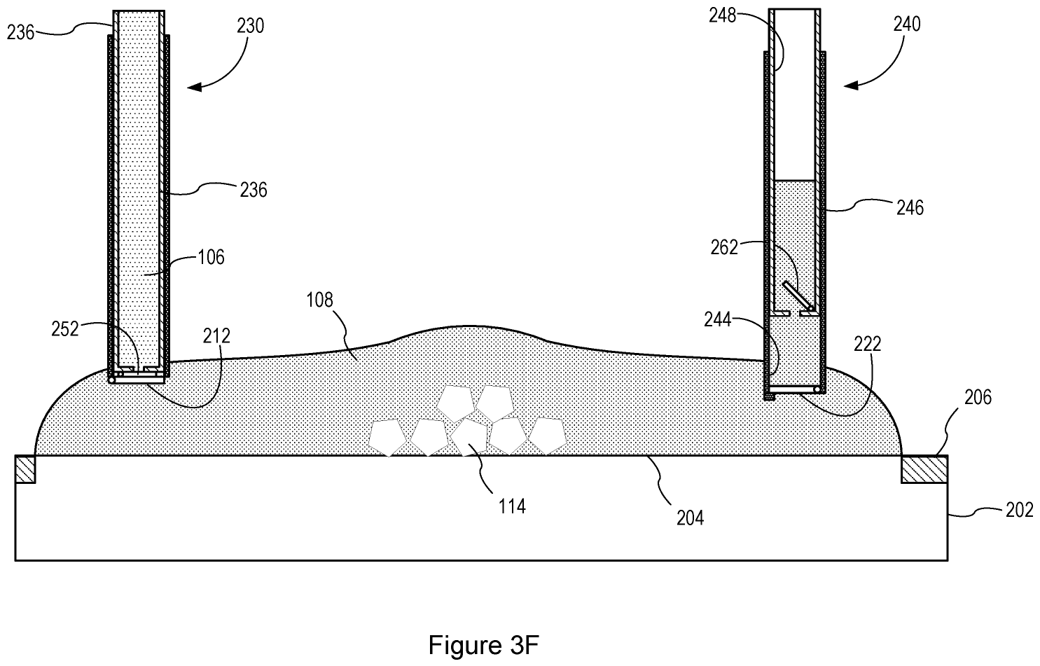

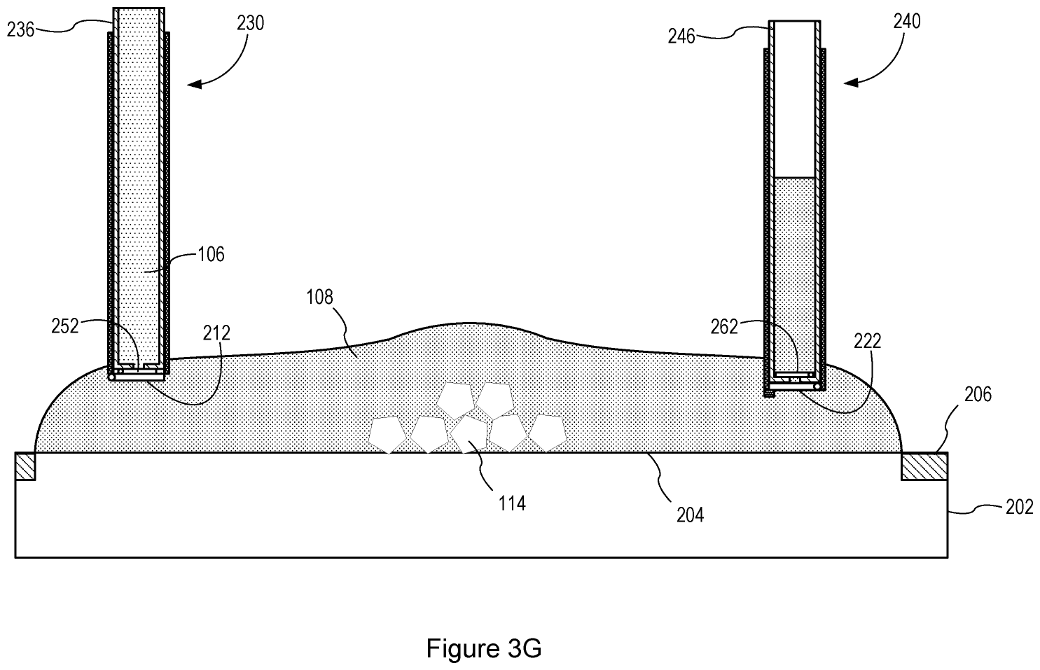

FIG. 2D is similar to FIG. 2C, except that piston 236 defines channel 238 within piston 236 and piston 236 is coupled with valve 252 (e.g., a one-way valve, a check valve, etc.), and piston 246 defines channel 248 within piston 246 and piston 246 is coupled with valve 262 (e.g., a one-way valve, a check valve, etc.). Channel 238 and valve 252 are configured to deliver a precise volume of wash liquid 106 into channel 234. Channel 248 and valve 262 are configured to remove mixture 108 in channel 244. The operations of these components are described further below with respect to FIGS. 3A-3G.

FIG. 2E is similar to FIG. 2D, except that filter 250 is coupled with a tip of aspirator 240. In some implementations, filter 250 reduces or prevents aspiration of cells. In some embodiments, filter 250 has a plurality of pores. In some embodiments, the plurality of pores has a pore size between 0.1 and 20 μm. In some embodiments, the plurality of pores has a pore size between 1 and 10 μm. In some embodiments, the plurality of pores has a pore size between 1 and 5 μm. In some embodiments, the plurality of pores has a pore size between 2 and 8 μm.

FIG. 2E also illustrates that aspirator 240 is coupled with vibrator 254. In FIG. 2E, vibrator 254 is positioned adjacent to filter 250. Vibrator 254 is configured to provide vibration to filter 250, which reduces clogging of filter 250 by preventing accumulation of cells on filter 250. In some embodiments, vibrator 254 is a piezo-electric vibrator.

FIG. 3A illustrates that dispenser 230 includes piston 236 in a first position. The channel defined within piston 236 includes wash liquid 106.

FIG. 3B illustrates that piston 236 moves up to a second position, which allows liquid 106 in the channel defined within piston 236 to flow into channel 234. During the upward movement of piston 236, there is a negative pressure within channel 234, which keeps valve 212 closed.

Once channel 234 is filled with a predefined volume of wash liquid 106, piston 236 moves down to push wash liquid 106 out of channel 234. FIG. 3C illustrates that piston 236 moves down, which causes valve 252 to close. The increased pressure within channel 234 opens valve 212 so that wash liquid 106 in channel 234 is dispensed (e.g., released) into sample solution 104, thereby forming mixture 108.

FIG. 3D illustrates that piston 236 has returned to the first position. In FIG. 3D, piston 246 of aspirator 240 is in a third position.

FIG. 3E illustrates an upward movement of piston 246 to a fourth position. The negative pressure within channel 244 causes valve 222 to open, which allows a portion of mixture 108 to flow into channel 244. The negative pressure within channel 244 causes valve 262 to close so that mixture 108 does not flow into the channel 248.

Once channel 244 is filled with a predefined volume of mixture 108, piston 246 moves down to move mixture 108 in channel 244 to channel 248. FIG. 3F illustrates piston 246 moves down, which causes valve 222 to close. The increased pressure within channel 244 opens valve 262 so that mixture 108 in channel 244 flows into channel 248.

FIG. 3G illustrates that piston 246 has returned to the third position.

In some embodiments, dispenser 230 is coupled with a wash liquid source (e.g., a reservoir containing a wash liquid, which is optionally combined with a pump configured to provide the wash liquid). For example, wash liquid 106 is provided to channel 238 by the wash liquid source. In some embodiments, aspirator 240 is coupled with a suction pump. For example, mixture 108 in channel 248 is removed by the suction pump. In some embodiments, aspirator 240 is coupled with a reservoir. For example, mixture 108 in channel 248 is drained to the reservoir while piston 246 moves up.

In some embodiments, subsequent to dispensing wash liquid 106 and prior to aspirating a portion of mixture 108, mixture 108 is shaken and/or agitated (e.g., the array plate on which mixture 108 is located is shaken and/or agitated by placing the array plate on a shaker and activating the shaker).

In some embodiments, one or more valves illustrated in FIGS. 3A-3G (e.g., valves 212, 222, 252, and 262) are spring-loaded. A spring-loaded valve is configured to close itself and/or remain closed when a pressure difference applied on the valve is less than a predefined threshold.

Although FIGS. 3A-3G illustrate that a single dispenser and a single aspirator for a single sample spot, in some embodiments, multiple dispensers and/or multiple aspirators are used for a single sample spot (e.g., using multiple dispensers and multiple aspirators for a particular sample spot can reduce the washing time, especially for a large sample spot). In some embodiments, multiple dispensers are configured for concurrent operations and/or multiple aspirators are configured for concurrent operations.

In some embodiments, a single dispenser is used for dispensing a wash liquid into multiple spots. For example, a single dispenser is coupled with a split channel (e.g., 2-channel, 4-channel, 8-channel, 12-channel, 16-channel, 32-channel, 64-channel, 128-channel, 256-channel splitter). In some embodiments, a single aspirator is used for aspirating liquid (e.g., a mixture) from multiple spots. For example, a single aspirator is coupled with a split channel (e.g., 2-channel, 4-channel, 8-channel, 16-channel, 32-channel, 64-channel, 128-channel, 256-channel splitter).

In some embodiments, one or more of a dispenser and an aspirator are coupled with a positive displacement pump (e.g., a membrane pump, such as a solenoid micropump). The positive displacement pump reduces the variation in the volume of the dispensed liquid or the volume of the aspirated liquid. In some embodiments, a dispenser is coupled with a positive displacement pump without a valve. In some embodiments, an aspirator is coupled with a positive displacement pump without a valve.

Although FIGS. 2A-2E and 3A-3G illustrate configurations, in which both a dispenser and an aspirator are concurrently in contact with a liquid (e.g., solution 104 or mixture 108), a person having ordinary skill in the art would understand that only one of the dispenser and the aspirator may be in contact with the liquid (e.g., a dispenser comes in contact with solution 104 first for dispensing a wash liquid, while an aspirator remains separated from solution 104, and the dispenser is subsequently removed from mixture 108 of solution 104 and the wash liquid, and the aspirator comes in contact with mixture 108 for aspirating a portion of mixture 108 while the dispenser remains separated from mixture 108). In some embodiments, a dispenser is used at a first time without an aspirator, and an aspirator is used at a second time distinct from the first time (e.g., the second time is subsequent to the first time) without a dispenser. For brevity, these details are omitted.

In FIGS. 1A-1F, 2A-2E, and 3A-3G, top portions of dispensers and aspirators are truncated to simplify the drawings.

Although FIGS. 2A-2E and 3A-3G illustrate washing operations, analogous operations can be used for introducing reagents to the array plate (or the cells on the array plate). For example, instead of a wash liquid, a reagent liquid (e.g., a liquid containing reagents for reaction with cells) is used in some implementations. Such operations can introduce the reagents without agitating the cells on the array plate, thereby improving the accuracy and reliability of reaction between the reagents and the cells. In addition, the loss of the cells is reduced by using such operations.

Although FIGS. 2A-2E and 3A-3G illustrate an aspirator located away from a dispenser (e.g., the aspirator and the dispenser are located toward two opposite ends of solution 104), in some implementations, the aspirator and the dispenser are located adjacent to each other (e.g., the aspirator and the dispenser are located toward a same end of solution 104, or toward the center of solution 104).

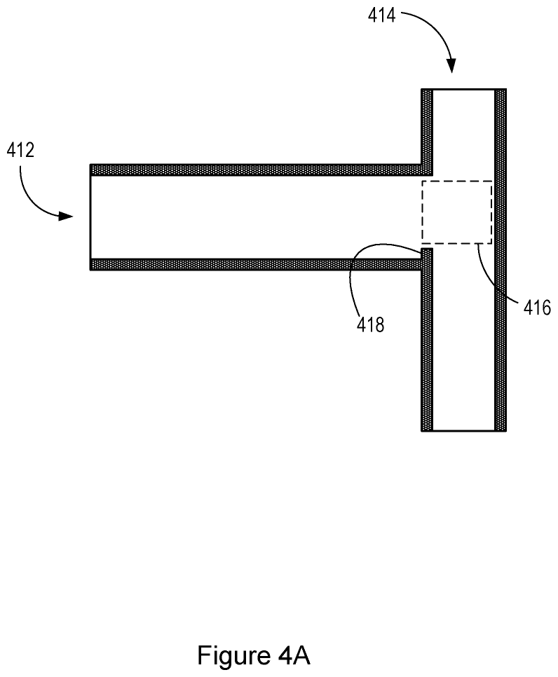

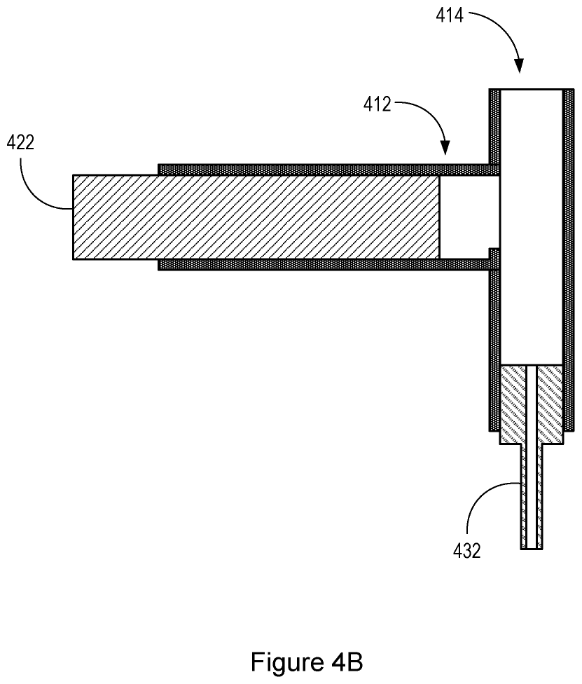

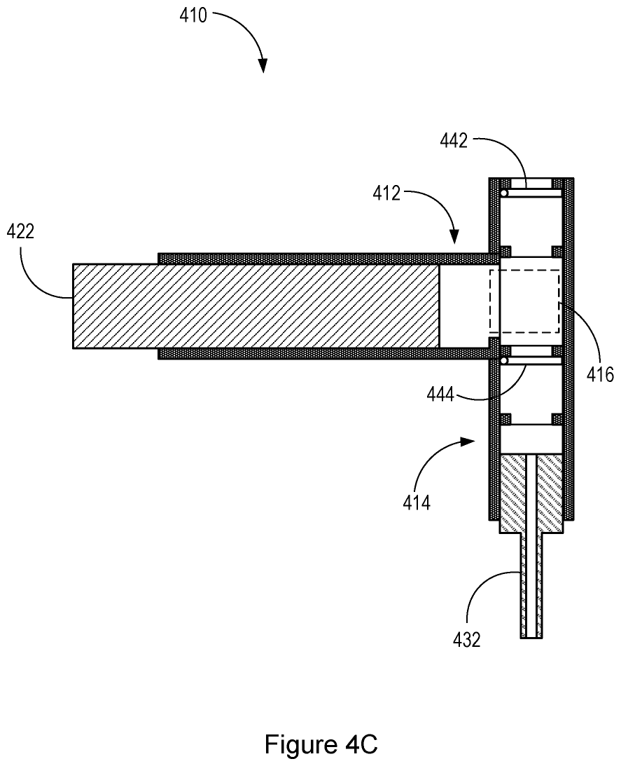

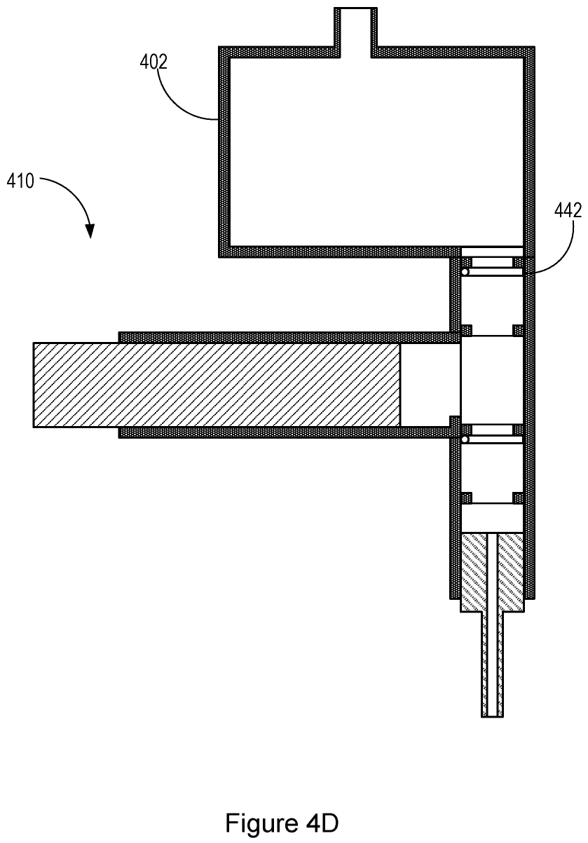

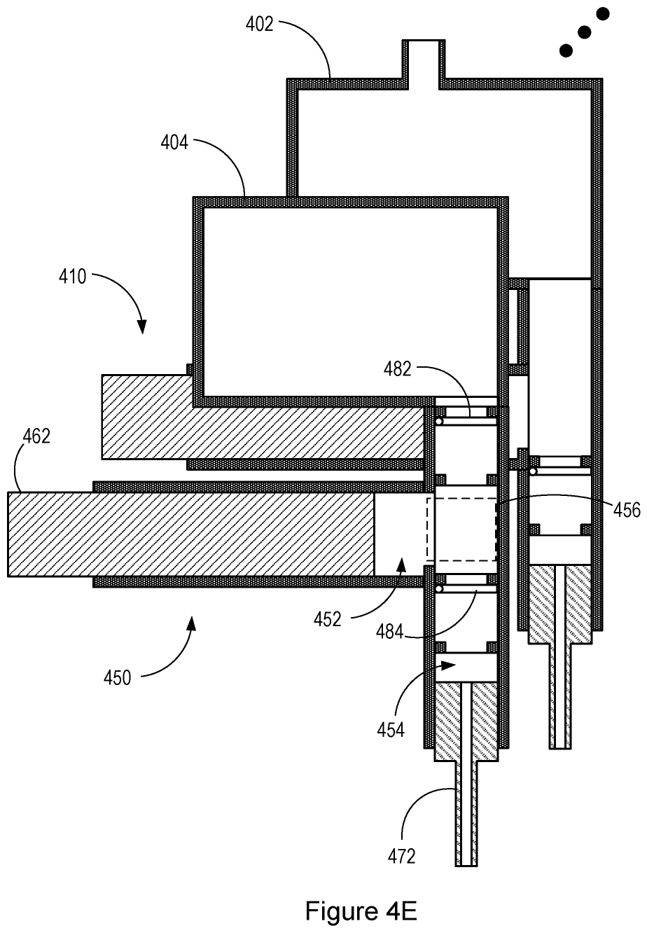

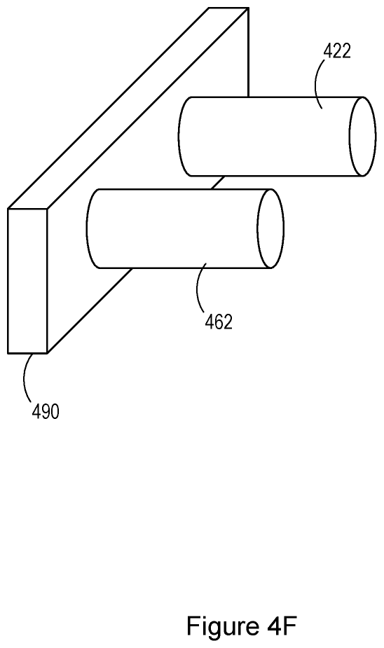

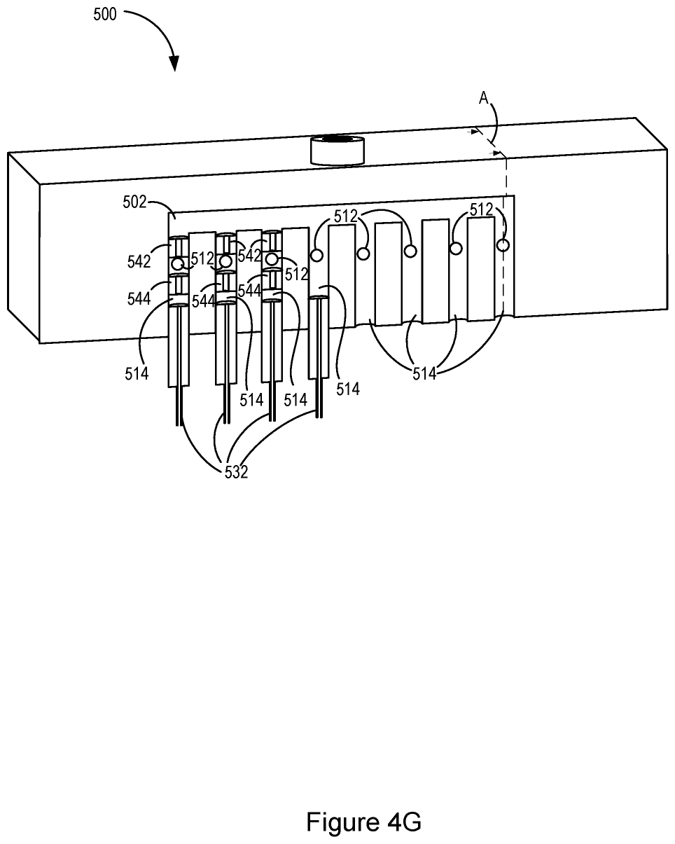

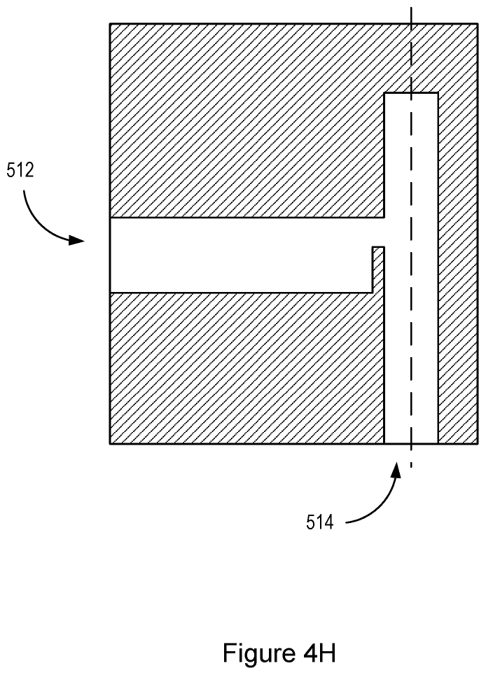

FIGS. 4A-4C illustrate a dispenser and its components in accordance with some embodiments.

FIG. 4A illustrates a first piston channel 412 and a first nozzle channel 414 connected to the first piston channel 412 at a first intersection 416. The first nozzle channel 414 is non-parallel to the first piston channel 412 (e.g., the first nozzle channel 414 and the first piston channel 412 form an angle that is between 30 and 150 degrees). In some embodiments, the first nozzle channel 414 is substantially perpendicular to the first piston channel 412 (e.g., the first nozzle channel 414 and the first piston channel 412 form an angle that is between 75 and 105 degrees).

In some embodiments, at least one of the first nozzle channel 414 and the first piston channel 412 include a stopper to prevent movement of a piston into the first nozzle channel 414.

In some embodiments, the first piston channel 412 is defined in a first barrel (or a pipe or a tube). In some embodiments, the first nozzle channel 414 is defined in a second barrel (or a pipe or a tube). In some embodiments, the first barrel and the second barrel are integrally formed. In some embodiments, the first barrel and the second barrel are separate barrels that are coupled together.

FIG. 4B illustrates that a first piston 422 is located at least partially within the first piston channel 412. The first piston 422 is configured to slide within the first piston channel 412 (e.g., an outer diameter of the first piston 422 is less than the diameter of the first piston channel, such as an inner diameter of the first barrel defining the first piston channel 412). In some embodiments, the first piston 422 is coupled with one or more seals (e.g., o-rings) to prevent leakage through a gap between the first piston 422 and the first piston channel 412.

FIG. 4B also illustrates that a first nozzle 432 is coupled with the first nozzle channel 414. In some embodiments, the first nozzle 432 includes one or more seals (e.g., o-rings) to prevent leakage through a gap between the first nozzle 432 and the first nozzle channel 414.

FIG. 4C illustrates a first dispenser 410 in accordance with some embodiments. The first dispenser 410 includes, in addition to the components shown in FIG. 4B, a first valve 442 (e.g., a first one-way valve) and a second valve 444 (e.g., a second one-way valve). In some embodiments, the first valve 442 is located above the first intersection 416 in the first nozzle channel 414 and the second valve 444 is located below the first intersection 416 in the first nozzle channel 414, as shown in FIG. 4C. In some embodiments, the first valve 442 and the second valve 444 are located adjacent to the first intersection 416. In some embodiments, the first valve 442 and the second valve 444 are located away from the first intersection 416.

FIG. 4D illustrates the first dispenser 410 coupled with a reservoir 402 in accordance with some embodiments. In some embodiments, the reservoir 402 is a dispensing-liquid reservoir containing liquid that is to be dispensed through the first dispenser 410. In some embodiments, the reservoir 402 has an outlet that is coupled toward the first valve 442 of the first dispenser 410. In some embodiments, the reservoir 402 has one or more inlets. In some embodiments, at least one of the one or more inlets is coupled with a pump or an input line so that the reservoir 402 can receive additional liquid. In some embodiments, at least one of the one or more inlets is exposed to an ambient environment (e.g., air) so that the pressure inside the reservoir 402 corresponds to the atmospheric pressure.

FIG. 4E illustrates a set of multiple dispensers in accordance with some embodiments. The set of multiple dispensers includes the first dispenser 410 and a second dispenser 450.

The second dispenser 450 has a second piston channel 452 and a second nozzle channel 454 that is connected to the second piston channel 452 at a second intersection 456. In some embodiments, the second nozzle channel 454 is non-parallel to the second piston channel 452.

The second dispenser 450 includes a second piston 462, a second nozzle 472, a third valve 482, and a fourth valve 484. These components are similar to the first piston 412, the first nozzle 432, the first valve 442, and the second valve 444, respectively. For brevity, the description of these components is omitted herein. However, a person having ordinary skill in the art would understand their structures and operations based on the description of the corresponding components, namely the first piston 412, the first nozzle 432, the first valve 442, and the second valve 444, as described herein.

In FIG. 4F, the second dispenser 450 is coupled with a second reservoir 404 that is distinct and separate from the first reservoir 402. In some embodiments, both the first dispenser 410 and the second dispenser 450 are coupled with the same reservoir 402.

FIG. 4F illustrates coupled pistons in accordance with some embodiments. In FIG. 4F, the first piston 422 and the second piston 462 are mechanically coupled to a common holder 490 (which may have the shape of a plate, a rod, a block, etc.) so that a movement of the common holder 490 causes concurrent movement of the first piston 422 and the second piston 462.

In some embodiments, the common holder 490 is coupled to an actuator, which causes a movement of the common holder 490. In some embodiments, the actuator includes a motor (e.g., a stepper motor, a DC motor, etc.), a linear actuator, etc.

FIG. 4G illustrates a sectional view of a block with piston channels and nozzle channels in accordance with some embodiments. Although FIGS. 4A-4F illustrate the first piston channel 412 and the first nozzle channel 414 defined by one or more barrels (e.g., the first piston channel 412 corresponds to a hollow space within a first barrel and the second nozzle channel 414 corresponds to a hollow space within a second barrel), the first piston channel 412 and the first nozzle channel 414 may be defined by using other parts. For example, as shown in FIG. 4G, piston channels 512 and nozzle channels 514 are defined in a block 500. In some embodiments, the block 500 is a single integrated block (e.g., a single piece of a particular material, such as metal, ceramics, plastic, or a composite material) with a cavity to define the piston channels 512 and the nozzle channels 514. In such embodiments, the block 500 may be made by casting (e.g., metal casting), machining, three-dimensional printing, any combination thereof, etc. In some other embodiments, the block 500 is a combination of multiple parts (e.g., one or more plates and/or sub-blocks) that are assembled together to define the piston channel 512 and the nozzle channels 514.

In some embodiments, the block 500 also defines a reservoir 502. In some embodiments, as shown in FIG. 4G, the reservoir 502 is coupled to the plurality of nozzle channels 514. In some embodiments, the block 500 may define multiple reservoirs, including a first reservoir coupled to a first subset of the nozzle channels 514 and a second reservoir coupled to a second subset of the nozzle channels 514 that is different from and mutually exclusive to the first subset of the nozzle channels 514.

FIG. 4G also shows a plurality of nozzles 532, a plurality of first valves 542, and a plurality of second valves 544, located in respective nozzle channels 514. In FIG. 4G, the nozzles 532, the first valves 542, and the second valves 544 are not shown for every nozzle channel 514 so as not to obscure other aspects of the block 500. However, a person having ordinary skill in the art would understand that each nozzle channel 514 may contain a respective nozzle 532, a respective first valve 542, and a respective second valve 544.

Also shown in FIG. 4G is line A, from which the cross-sectional view of the block 500, shown in FIG. 4H, is taken. FIG. 4H illustrates the piston channel 512 perpendicularly connected to the nozzle channel 514.

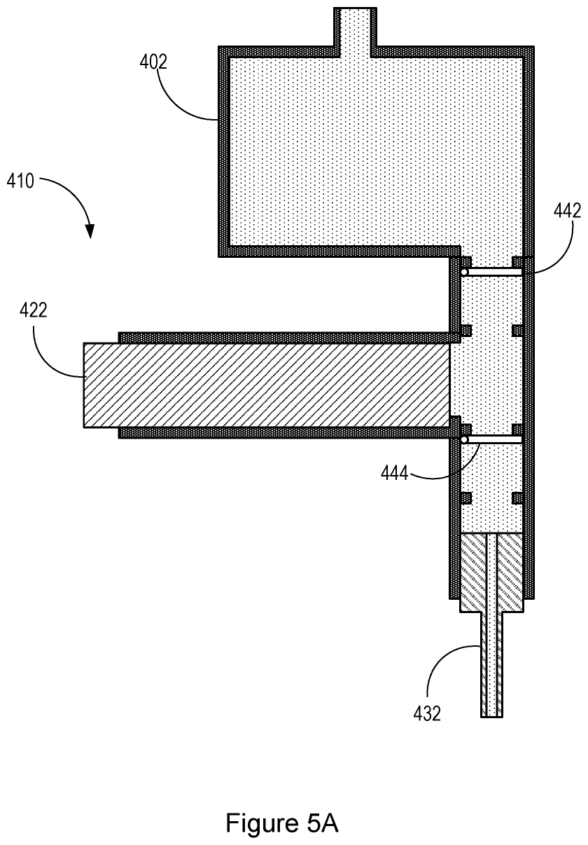

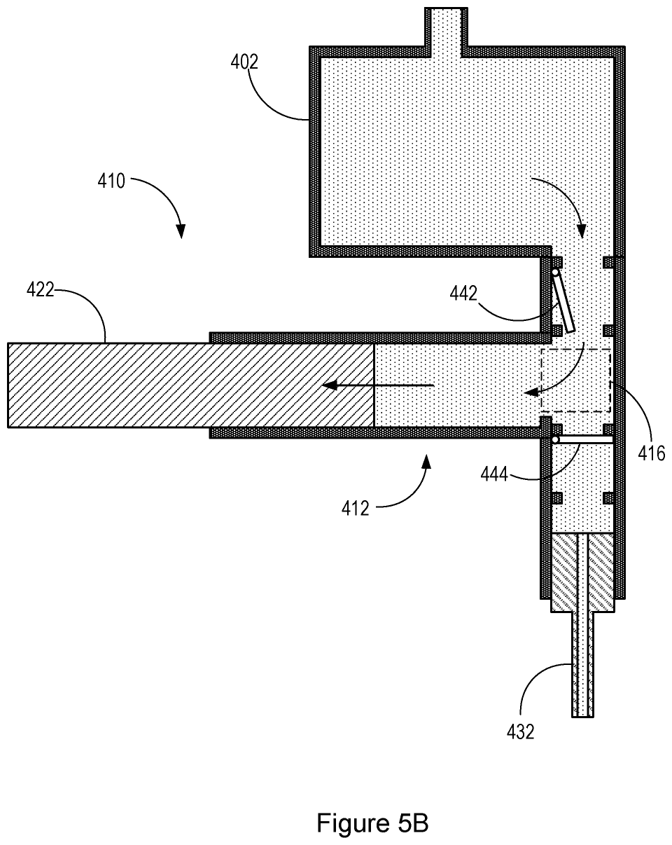

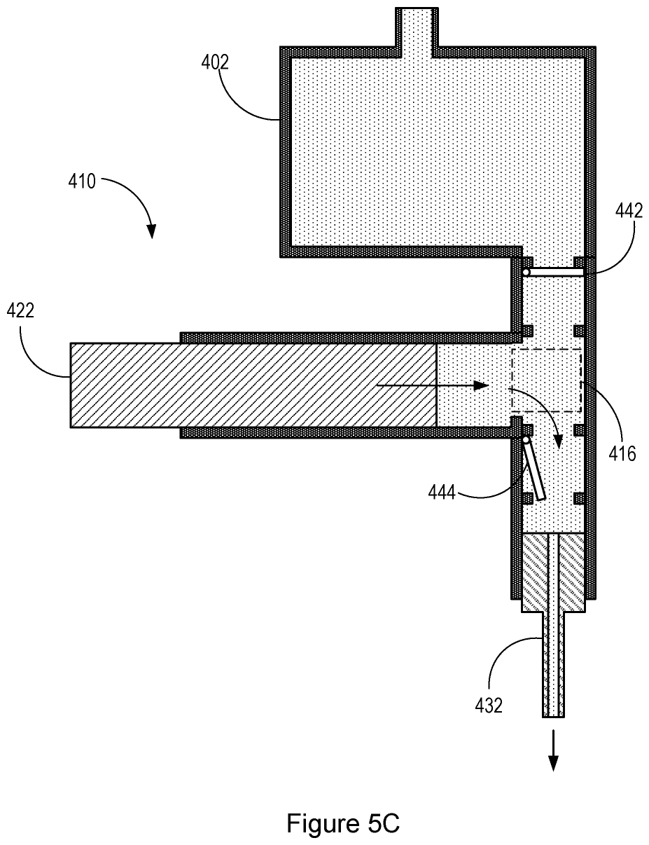

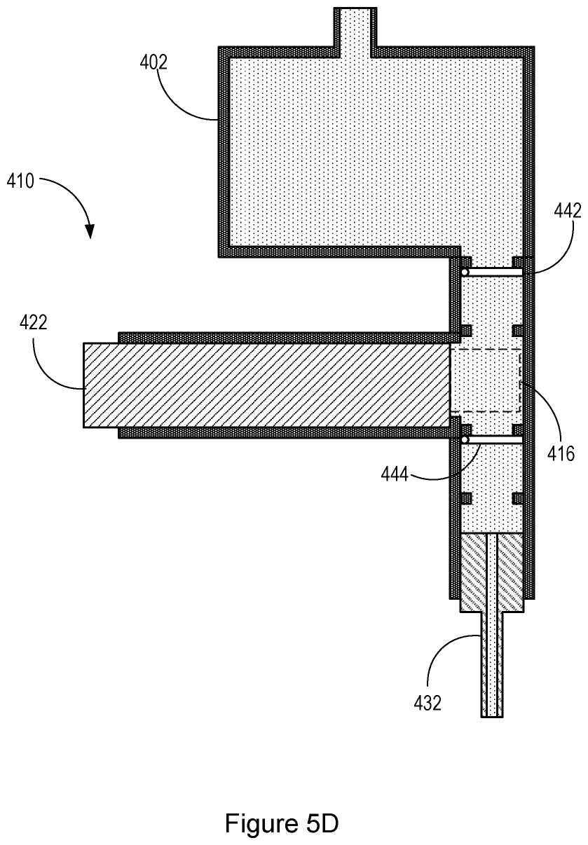

FIGS. 5A-5D illustrate a dispensing operation in accordance with some embodiments.

FIG. 5A illustrates the dispenser 410 coupled to the reservoir 402 containing a liquid (e.g., a wash solution).

FIG. 5B illustrates that the first piston 422 of the dispenser 410 is pulled, which initiates a flow of the liquid in the reservoir 402 to the first intersection 416 (by providing a pressure differential between the reservoir 402 and the first intersection 416, which, in turn, opens the first valve 442). In some cases, the liquid also flows into the first piston channel 412, as shown in FIG. 5B. Pulling the first piston 422 also decreases the pressure within the first intersection 416 (relative to the pressure around an inlet of the first nozzle 432) so that the second valve 444 remains closed.

FIG. 5C illustrates that the first piston 422 is pushed, which initiate a flow of the liquid in the first intersection 416 (and optionally, the liquid in the first piston channel 412) to the first nozzle 432 (by providing a pressure differential between the first intersection 416 and an inlet of the first nozzle 432, which, in turn, opens the second valve 444) so that the liquid is dispensed from the first nozzle 432. Pushing the first piston 422 also increases the pressure within the first intersection 416 (relative to the pressure within the reservoir 402) so that the first valve 442 remains closed.

FIG. 5D illustrates that, in some cases, after the first piston 422 ceases to move (e.g., the first piston 422 is neither pulled nor pushed), the pressure within the first intersection 416 corresponds to the pressure around the inlet of the first nozzle 432 so that the second valve 444 closes. In some cases, at least one of the first valve 442 and the second valve 444 includes an elastic object (e.g., a spring), which facilitates (or accelerates) closing of the valve.

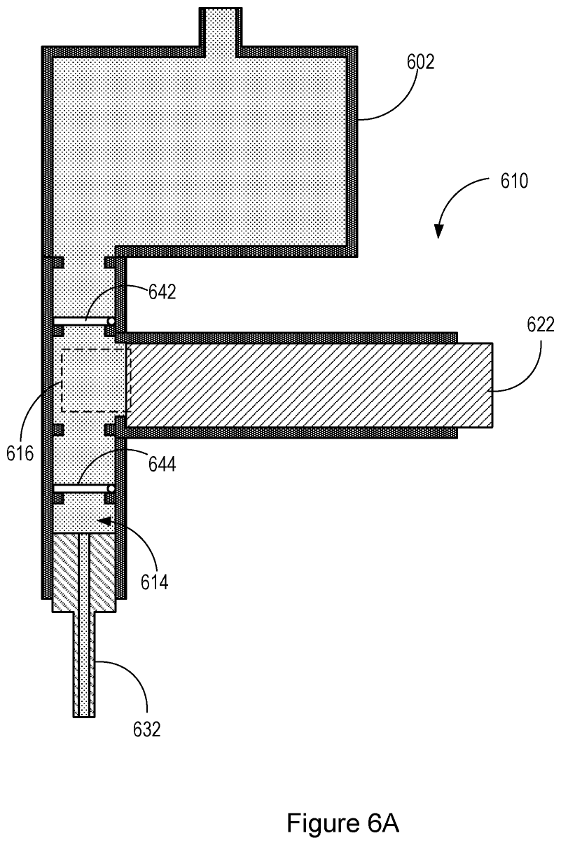

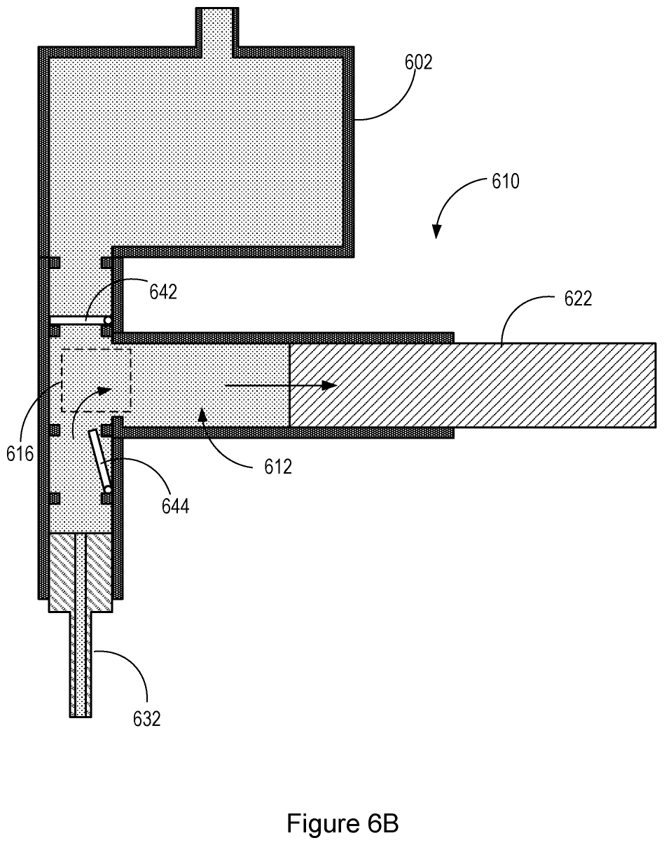

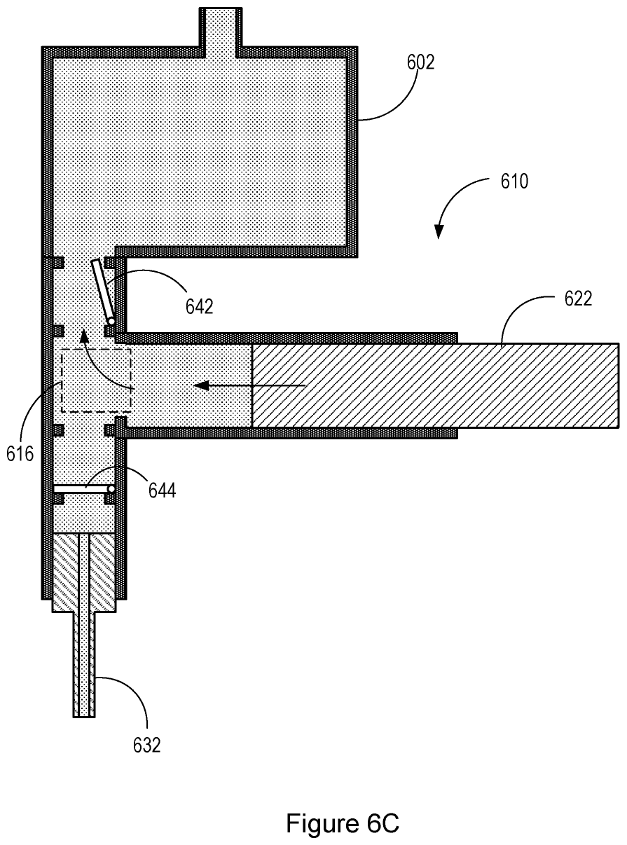

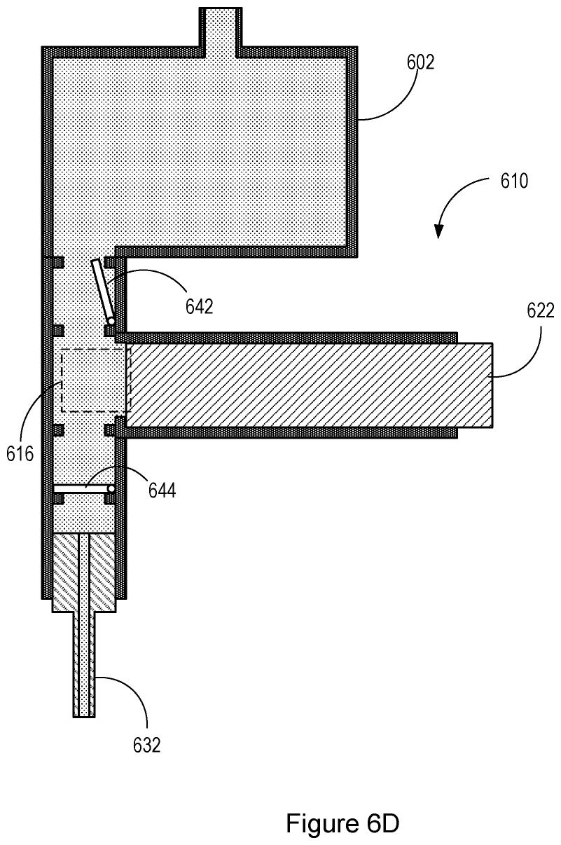

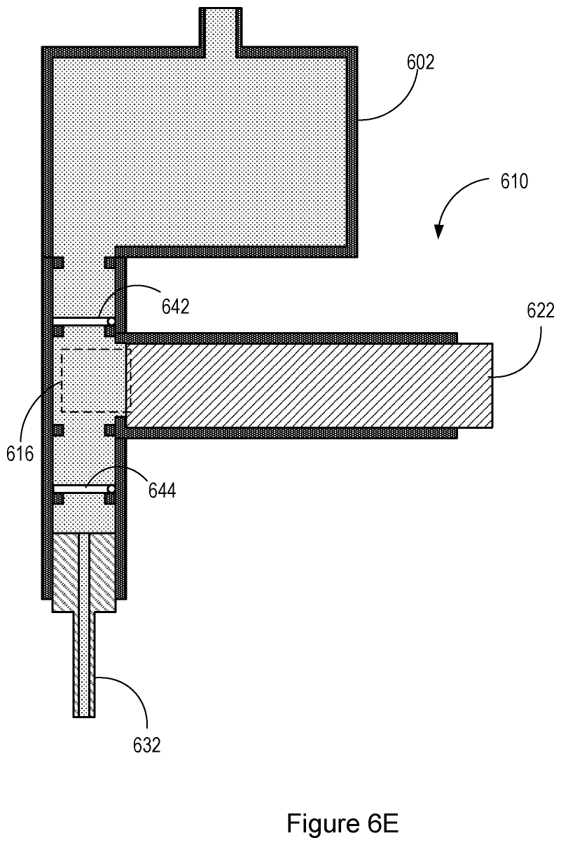

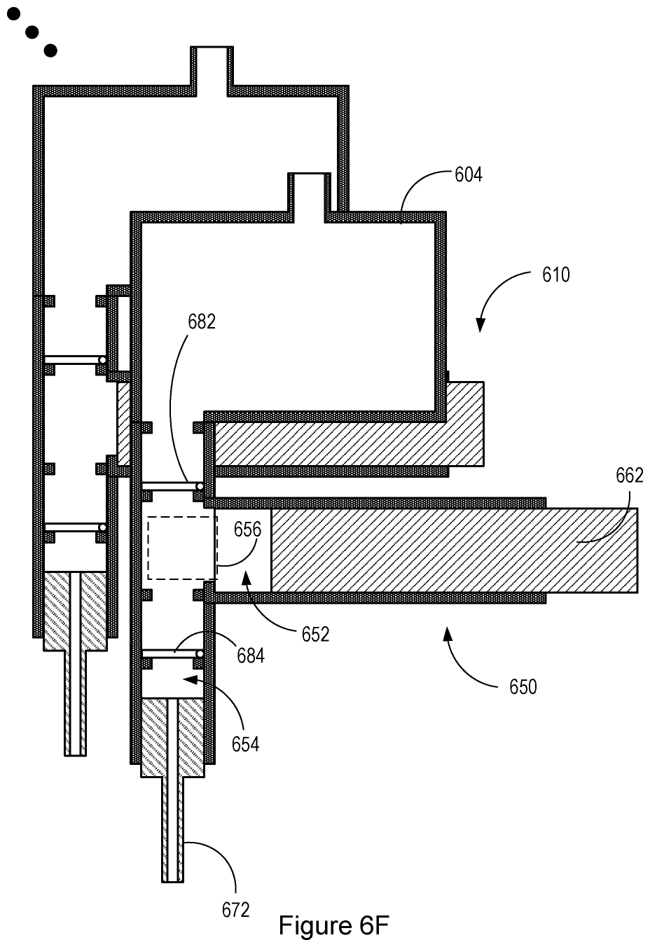

FIGS. 6A-6E illustrate an aspiration operation in accordance with some embodiments.

FIG. 6A illustrates an aspirator 610 coupled to a reservoir 602 (e.g., an aspirated-liquid reservoir) configured to receive (and optionally store) an aspirated liquid (e.g., a mixture of a sample solution and a wash solution). The aspirator 610 has a first piston channel and a first nozzle channel 614 coupled to the first piston channel. The aspirator 610 includes a first piston 622, a first nozzle 632, a first valve 642, and a second valve 644. The first piston 622, the first nozzle 632, the first valve 642, and the second valve 644 correspond to the first piston 422, the first nozzle 432, the first valve 442, and the second valve 444 of the first dispenser 410, except that the directionality of the first valve 442 and the second valve 444 is reversed.

FIG. 6B illustrates that the first piston 622 of the aspirator 610 is pulled, which initiates a flow of the liquid in the first nozzle 632 to the first intersection 616 (by providing a pressure differential between the first nozzle 632 and the first intersection 616, which, in turn, opens the second valve 644). This also facilitates aspirating liquid adjacent to the first nozzle 632 into the first intersection 616. In some cases, the liquid also flows into the first piston channel 612, as shown in FIG. 6B. Pulling the first piston 622 also decreases the pressure within the first intersection 616 (relative to the pressure in the reservoir 602) so that the first valve 642 remains closed.

FIG. 6C illustrates that the first piston 622 is pushed, which initiate a flow of the liquid in the first intersection 616 (and optionally, the liquid in the first piston channel 612) to the reservoir 602 (by providing a pressure differential between the first intersection 616 and the reservoir 602, which, in turn, opens the first valve 642). Pushing the first piston 622 also increases the pressure within the first intersection 616 (relative to the pressure within the first nozzle 632) so that the second valve 642 remains closed.

FIGS. 6D and 6E illustrate that, in some cases, after the first piston 622 ceases to move (e.g., the first piston 622 is neither pulled nor pushed), the pressure within the first intersection 616 corresponds to the pressure in the reservoir 602 so that the first valve 642 closes. In some cases, at least one of the first valve 642 and the second valve 644 includes an elastic object (e.g., a spring), which facilitates (or accelerates) closing of the valve.

FIG. 6F illustrates a set of multiple aspirators in accordance with some embodiments. The set of multiple aspirators includes the first aspirator 610 and a second aspirator 650.

The second aspirator 650 has a second piston channel 652 and a second nozzle channel 654 that is connected to the second piston channel 652 at a second intersection 656. In some embodiments, the second nozzle channel 654 is non-parallel to the second piston channel 652.

The second aspirator 650 includes a second piston 662, a second nozzle 672, a third valve 682, and a fourth valve 684. These components are similar to the first piston 612, the first nozzle 632, the first valve 642, and the second valve 644, respectively. For brevity, the description of these components is omitted herein. However, a person having ordinary skill in the art would understand their structures and operations based on the description of the corresponding components, namely the first piston 612, the first nozzle 632, the first valve 642, and the second valve 644, as described herein.

In some embodiments, the piston channels and the nozzle channels of aspirators are defined using barrels. In some embodiments, the piston channels and the nozzle channels of aspirators are defined in a block (e.g., the block 500).

Although the dispensers 410 and 460 are illustrated separately from the aspirators 610 and 650, a person having ordinary skill in the art would understand that the dispensers 410 and 450 may be used in conjunction with the aspirators 610 and 650, in a manner analogous to those described with respect to FIGS. 2A-2E and 3A-3G.

In some embodiments, a block defines piston channels and nozzle channels for both dispensers and aspirators. This enables a compact washing device.

The foregoing description, for purpose of explanation, has been described with reference to specific embodiments. However, the illustrative discussions above are not intended to be exhaustive or to limit the embodiments to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

1-10. (canceled)

11. A device, comprising:
a first dispenser defining a first piston channel and a first nozzle channel that is non-parallel to the first piston channel and is connected to the first piston channel at a first intersection, the first dispenser including:
a first piston configured to slide at least partially within the first piston channel;
a first nozzle coupled with the first nozzle channel;
a first valve located between a dispensing-liquid reservoir and the first intersection, the first valve configured to allow a liquid in the dispensing-liquid reservoir to flow to the first intersection through the first valve while preventing a liquid in the first intersection from flowing to the dispensing-liquid reservoir through the first valve; and
a second valve located between the first intersection and the first nozzle, the second valve configured to allow a liquid in the first intersection to flow to the first nozzle through the second valve while preventing a liquid in the first nozzle from flowing to the first intersection through the second valve.

12. The device of claim 11, further comprising:
a second dispenser defining a second piston channel and a second nozzle channel that is non-parallel to the second piston channel and is connected to the second piston channel at a second intersection, the second dispenser including:
a second piston configured to slide at least partially within the second piston channel;
a second nozzle coupled with the second nozzle channel;
a third valve located between the dispensing-liquid reservoir and the second intersection, the third valve configured to allow a liquid in the dispensing-liquid reservoir to flow to the second intersection through the third valve and prevent a liquid in the second intersection from flowing to the dispensing-liquid reservoir through the third valve; and
a fourth valve located between the second intersection and the second nozzle, the fourth valve configured to allow a liquid in the second intersection to flow to the second nozzle through the fourth valve and prevent a liquid in the second nozzle to flow to the second intersection through the fourth valve.

13. The device of claim 12, wherein:
the first piston and the second piston are mechanically coupled to each other so that the first piston and the second piston are configured to move at a same speed in a same direction.

14. The device of claim 11, further comprising:
a first aspirator defining a third piston channel and a third nozzle channel that is non-parallel to the third piston channel and is connected to the third piston channel at a third intersection, the first aspirator including:
a third piston configured to slide at least partially within the third piston channel;
a third nozzle coupled with the third nozzle channel;
a fifth valve located between an aspirated-liquid reservoir and the third intersection, the fifth valve configured to allow a liquid in the third intersection to flow to the aspirated-liquid reservoir through the fifth valve and prevent a liquid in the aspirated-liquid reservoir from flowing to the third intersection through the fifth valve; and
a sixth valve located between the third intersection and the third nozzle, the sixth valve configured to allow a liquid in the third nozzle to flow to the third intersection through the sixth valve and prevent a liquid in the third intersection from flowing to the third nozzle through the sixth valve.

15. The device of claim 14, further comprising:
a second aspirator defining a fourth piston channel and a fourth nozzle channel that is non-parallel to the fourth piston channel and is connected to the fourth piston channel at a fourth intersection, the second aspirator including:
a fourth piston configured to slide at least partially within the fourth piston channel;
a fourth nozzle coupled with the fourth nozzle channel;
a seventh valve located between the aspirated-liquid reservoir and the fourth intersection, the seventh valve configured to allow a liquid in the aspirated-liquid reservoir to flow to the fourth intersection through the seventh valve and prevent a liquid in the fourth intersection from flowing to the aspirated-liquid reservoir through the seventh valve; and
an eighth valve located between the fourth intersection and the fourth nozzle, the eighth valve configured to allow a liquid in the fourth intersection to flow to the fourth nozzle through the eighth valve.

16. The device of claim 15, wherein:
the third piston and the fourth piston are mechanically coupled to each other so that the third piston and the fourth piston are configured to move at a same speed in a same direction.

17. The device of claim 11, wherein:
the first piston channel is substantially perpendicular to the first nozzle channel.

18. A device, comprising:
a first aspirator defining a first piston channel and a first nozzle channel that is non-parallel to the first piston channel and is connected to the first piston channel at a first intersection, the first aspirator including:
a first piston configured to slide at least partially within the first piston channel;
a first nozzle coupled with the first nozzle channel;
a first valve located between an aspirated-liquid reservoir and the first intersection, the first valve configured to allow a liquid in the first intersection to flow to the aspirated-liquid reservoir through the first valve and prevent a liquid in the aspirated-liquid reservoir from flowing to the first intersection through the first valve; and
a second valve located between the first intersection and the first nozzle, the second valve configured to allow a liquid in the first nozzle to flow to the first intersection through the second valve and prevent a liquid in the first intersection from flowing to the first nozzle through the second valve.

19. The device of claim 18, further comprising:
a second aspirator defining a second piston channel and a second nozzle channel that is non-parallel to the second piston channel and is connected to the second piston channel at a second intersection, the second aspirator including:
a second piston configured to slide at least partially within the second piston channel;
a second nozzle coupled with the second nozzle channel;
a third valve located between the aspirated-liquid reservoir and the second intersection, the third valve configured to allow a liquid in the aspirated-liquid reservoir to flow to the second intersection through the third valve and prevent a liquid in the second intersection from flowing to the aspirated-liquid reservoir through the third valve; and
a fourth valve located between the second intersection and the second nozzle, the fourth valve configured to allow a liquid in the second intersection to flow to the second nozzle through the fourth valve.

20. The device of claim 19, wherein:
the first piston and the second piston are mechanically coupled to each other so that the first piston and the second piston are configured to move at a same speed in a same direction.

21. (canceled)

22. A device, comprising:
a first dispenser defining a first piston channel and a first nozzle channel that is connected to the first piston channel at a first intersection, the first dispenser including:
a first piston configured to slide at least partially within the first piston channel;
a first nozzle coupled with the first nozzle channel;
a first valve located between a dispensing-liquid reservoir and the first intersection, the first valve configured to allow a liquid in the dispensing-liquid reservoir to flow to the first intersection through the first valve while preventing a liquid in the first intersection from flowing to the dispensing-liquid reservoir through the first valve; and
a second valve located between the first intersection and the first nozzle, the second valve configured to allow a liquid in the first intersection to flow to the first nozzle through the second valve while preventing a liquid in the first nozzle from flowing to the first intersection through the second valve.
